(19) United States
(12) Patent Application Publication
(10) Pub. No.: US 2015/0079089 A1
(43) Pub. Date: Mar. 19, 2015

(54) EMP2 REGULATES ANGIOGENESIS IN CANCER CELLS THROUGH INDUCTION OF VEGF

(71) Applicant: THE REGENTS OF THE UNIVERSITY OF CALIFORNIA, Oakland, CA (US)

(72) Inventors: Madhuri Wadehra, Manhattan Beach, CA (US); Jonathan Braun, Tarzana, CA (US); Lynn K. Gordon, Tarzana, CA (US)

(21) Appl. No.: 14/376,724

(22) PCT Filed: Feb. 6, 2013
(86) PCT No.: PCT/US2013/024968

§ 371 Date: Aug. 5, 2014

Related U.S. Application Data
(60) Provisional application No. 61/595,617, filed on Feb. 6, 2012.

(51) Int. Cl. C07K 16/30; A61K 47/48; A61K 38/14; A61K 31/69; C07K 16/22; A61K 39/395

(52) U.S. Cl. CPC C07K 16/30; C07K 16/22; A61K 39/39558; A61K 38/14; A61K 31/69; A61K 47/48261; C07K 2317/24; C07K 2317/622; C07K 2317/34
USPC 424/135.1; 424/139.1; 424/133.1; 424/183.1

(57) ABSTRACT

Disclosed herein are methods and compositions for modulating neovascularization. The disclosed compositions find particular use in the treatment of uterine cancers, specifically endometrial cancers.

### CROSS-REFERENCES TO RELATED APPLICATIONS

This application claims priority benefit of U.S. Patent Application Ser. No. 61/595,617 filed Feb. 6, 2012, the disclosure of which is incorporated by reference in its entirety.

### STATEMENT AS TO GOVERNMENT SUPPORT

This invention was made with Government support under Grant Nos. CA16042 and CA131756 awarded by the National Institutes of Health, Grant No. CA-86366 awarded by the National Cancer Institute. The Government has certain rights in this invention.

This work was supported by the U.S. Department of Veterans Affairs, and the Federal Government has certain rights in this invention.

### REFERENCE TO SEQUENCE LISTING SUBMITTED ELECTRONICALLY

The sequence listing contained in the file named “008074-5050-WO_ST25.txt”, created on Feb. 6, 2013 and having a size of 32.2 kilobytes, has been submitted electronically herewith via EFS-Web, and the contents of the txt file are hereby incorporated by reference in their entirety.

### BACKGROUND

Cancer is a disease characterized by uncontrolled cell growth and proliferation of abnormal, cancer, cells derived from normal tissue. The uncontrolled growth and proliferation can lead to the cancer cells invading adjacent tissues. In some instances, the cancer cells can enter the lymphatic or circulatory system of an animal, spreading, i.e. metastasizing, to regional lymph nodes and to distant sites.

Many types of cancers are associated with angiogenesis, i.e. the process of new blood vessel formation from pre-existing vessels. Angiogenesis is a normal process, for example, during growth, development, wound healing, and in granulation tissue. However, it is also a fundamental step in tumorigenesis. In addition to its role in cancers, angiogenesis is implicated in the pathologies of numerous other diseases. For example, angiogenesis has been linked to proliferative retinopathies, age-related macular degeneration, rheumatoid arthritis (RA), and psoriasis.

There are two types of angiogenesis: intussusceptive angiogenesis and sprouting angiogenesis. Intussusceptive angiogenesis, also known as splitting angiogenesis, involves the repeated addition of transcapillary pillars in existing tumor vessels. In Intussusceptive angiogenesis, the capillary walls extend into the lumen and split a single vessel into two vessels. Sprouting angiogenesis the process by which new blood vessels grow from existing ones. Sprouting angiogenesis is a ubiquitous phenomenon in health and disease. It plays a pivotal role in diverse processes from embryo development to wound healing to tumor growth.

Regarding tumor angiogenesis, it has been shown that solid tumor growth depends on successful neovascularization and several factors. The most notable class of factors are the vascular endothelial growth factors (VEGFs). VEGFs were first described as a potent vascular permeability factor (VPF) secreted by tumor cells that stimulate a rapid and reversible increase in microvascular permeability without mast cell degranulation or endothelial cell damage. VEGF is a potent mitogen for vascular endothelial cells which has been reported as a pivotal regulator of both normal and abnormal angiogenesis. Ferrara and Davis-Smyth (1997) Endocrine Rev. 18:4-25; Ferrara (1999) J. Mol. Med. 77:527-543. Compared to other growth factors that contribute to the processes of vascular formation, VEGF is unique in its high specificity for endothelial cells within the vascular system. VEGF has also been suggested to be essential for embryonic vasculogenesis and angiogenesis. Carmeliet et al. (1996) Nature 380:435-439; Ferrara et al. (1996) Nature 380:439-442. Furthermore, VEGF is required for the cyclical blood vessel proliferation in the female reproductive tract and for bone growth and cartilage formation. Ferrara et al. (1998) Nature Med. 4:336-340; Gerber et al. (1999) Nature Med. 5:623-628.

In addition to being an angiogenic factor in angiogenesis and vasculogenesis, VEGF is a pleiotropic growth factor that exhibits multiple biological effects in physiological processes. For example, VEGF effects endothelial cell survival, vessel permeability and vasodilation, monocyte chemotaxis and calcium influx. Ferrara and Davis-Smyth (1997), supra. Recent studies also report mitogenic effects of VEGF on non-endothelial cell types, such as retinal pigment epithelial cells, pancreatic duct cells, and Schwann cells. Guerrin et al. (1995) J. Cell Physiol. 164:385-394; Oberg-Welsh et al. (1997) Mol. Cell. Endocrinol. 126:125-132; Sondell et al. (1999) J. Neurosci. 19:5731-5740.

VEGF has also been implicated in the disease development that involves pathological angiogenesis. For example, VEGF mRNA has been shown to be overexpressed in human tumors. Berkman et al. J Clin Invest 91:153-159 (1993); Brown et al. Human Pathol. 26:86-91 (1995); Brown et al. Cancer Res. 53:4727-4735 (1993); Mattern et al. Brit. J. Cancer. 73:931-934 (1996); and Dvorak et al. Am J. Pathol. 146:1029-1039 (1995). Also, VEGF concentration in eye fluids is highly correlated to the presence of active proliferation of blood vessels in patients with diabetes and other ischemia-related retinopathies. Aiello et al. N. Engl. J. Med. 331:1480-1487 (1994). Furthermore, studies suggest choroidal neovascular membrane localization of VEGF in patients affected by age-related macular degeneration (AMD). Lopez et al. Invest. Ophtalmo. Vis. Sci. 37:855-868 (1996).

Because VEGF is a primary regulator of angiogenesis in pathological conditions has led to numerous attempts to block VEGF activities Inhibitory anti-VEGF receptor antibodies, soluble receptor constructs, antisense strategies, RNA aptamers against VEGF and low molecular weight VEGF receptor tyrosine kinase (RTK) inhibitors have been developed to interfere with VEGF signaling. Siemeister et al. Cancer Metastasis Rev. 17:241-248 (1998). Specifically, anti-VEGF antibodies have been shown to block the VEGF cascade and suppress the growth of a variety of human tumor cell lines. Kim et al. Nature 362:841-844 (1993); Warren et al. J. Clin. Invest. 95:1789-1797 (1995); Borgstrom et al. Cancer Res. 56:4032-4039 (1996); and Melnyk et al. Cancer Res. 56:921-924 (1996). Furthermore, anti-VEGF antibodies have also been shown to inhibit intraocular angiogenesis in models of ischemic retinal disorders. Adamis et al. Arch. Opthalmol. 114:66-71 (1996). Accordingly, anti-VEGF antibodies such as bevacizumab (Avastin®; Genentech), a humanized anti-VEGF antibody, have been widely studied and used in the treatment of tumors and VEGF-associated disorders. VEGF polypeptides belong to the PDGF family of growth factors and are perhaps the most important players that regulate vessel formation. VEGFs are encoded by a family of genes that includes VEGF-A, -B, -C, -D and Placental growth factor (P1GF). They are dimeric cysteine-linked secreted glycoproteins with a molecular weight of ˜40 kDa. Produced in response to hypoxia, specific growth and differentiation factors, and by oncogenes, VEGFs are produced by many cell types including tumor cells. In tumors, VEGF-A appears to be the most potent angiogenic factor of the vascular growth factors, and its secretion has been shown to be critical for tumor growth. Thus, understanding the mechanisms that control angiogenesis, and in particular that control VEGF-A expression, are of paramount importance in tumor biology.

It has been suggested that focal adhesion kinase (FAK) signal transduction is implicated in VEGF production. FAK is a non-receptor protein-tyrosine kinase localized to cell substratum-extracellular matrix (ECM). In adherent cells, FAK is often associated with integrins at focal adhesions. Schaller et al. (1992) Proc. Natl. Acad. Sci. USA 89:5192-5196. Numerous other signaling proteins, including other protein tyrosine kinases are associated with FAK at these regions. Phosphorylation of FAK results in activation of the mitogen-activated protein kinase pathway. In addition, FAK regulates activation of phosphatidylinositol 3′-kinase which may serve to prevent apoptosis.

Overexpression of FAK is involved in cancer progression. For example, high levels of FAK correlate with invasiveness and metastatic potential in colon tumors, breast tumors, and oral cancers. Weiner et al. (1993) Lancet 342:1024-1025; Owens et al. (1995) Cancer Res., 55:2752-2755); Kornberg (1998) Head Neck 20:634-639. Furthermore, the role FAK plays in cell migration has led to the speculation that FAK plays a relevant role in the development and pathology of other diseases such as embryonic development and angiogenic disorders. Kornberg (1998) Head Neck 20:634-639.

It has been shown that epithelial membrane protein 2 (EMP2), a member of the tetraspan protein of the GAS-3/PMP22 family, regulates FAK and Src activation. EMP2 is expressed at high levels in epithelial cells of the lung, eye, and genitourinary tracts. Like several tetraspan proteins (CD9, CD81, PMP22), EMP2 in murine fibroblasts is localized to lipid raft domains. EMP2 controls cell surface trafficking and function of certain integrins, GPI-linked proteins, and class I MHC molecules, and reciprocally regulates caveolin expression. Claas et al. (2001) J Biol Chem 276:7974-84; Hasse et al. (2002) J Neurosci Res 69:227-32; Wadehra et al. (2003) Exp Mol Pathol 74:106-12; Wadehra et al. (2004) Mol Biol Cell 15:2073-2083; Wadehra et al. (2002) J Biol Chem 277:41094-41100; Wadehra et al. (2003) Clin Immunol 107:129-136.

Endometrial cancer (EC) is the most common gynecological malignancy. In the United States, the death rate from EC has doubled in the last twenty years, and currently a woman has approximately a 3% chance of developing EC during her lifetime. Silverberg et al. (2003) World Health Organization Classification of Tumors: Tumors of the Breast and Female Genital Tract, Lyon: IARC Press, p. 221-5′7; Sorosky (2008) Obstet Gynecol 111:436-47. EC is classified into two major sub-groups based on histology, clinical behavior, and epidemiology. The more common Type I is associated with estrogen predominance and pre-malignant endometrial hyperplasia. Hecht et al. (2006) J Clin Oncol 24:4783-91; Sherman (2000) Mod Pathol 13:295-308. Type II is mediated by non-hormonal risk factors, and often has a high grade or high-risk histology with an aggressive clinical. Hecht et al. (2006) J Clin Oncol 24:4783-91. Incidence of ECs generally increases with age, with 75-80% of new cases occurring in postmenopausal women. Creasman (1997) Semin Oncol 24:S1-140-S1-50.

Primary treatment for ECs is the surgical removal of the tumor, but recurrence is common, and other therapeutic interventions (radiotherapy, chemotherapy, and endocrine therapy) benefit only a subset of patients. Markman (2006), Semin Oncol 33: S33-8; Engleman et al. (2003) Semin Oncol 30:80-94. Presently, there are few biomarkers that distinguish ECs at the pre-malignant stage, although emerging efforts are targeting molecules that underlie the process of tumorigenesis. Kelloff et al. (2006) Clin Cancer Res 12:3661-97; Gossett et al. (2004) Int J Gynecol Cancer 14:145-51. Similarly, there are currently no biomarkers that can be targeted for tumor suppression and elimination. Thus, new modalities for early detection and treatment of ECs at premalignant and frankly malignant stages of disease are needed to improve management and prognosis.

One promising biomarker appears to be EMP2. EMP2 expression is associated with EMP2 neoplasia. Wadehra et al. (2006) Cancer 107:90-8. In endometrial cancer, EMP2 is an independent prognostic indicator for tumors with poor clinical outcome. EMP2 positive tumors, compared to EMP2 negative tumors, had a significantly greater myometrial invasiveness, higher clinical state, recurrent or persistent disease following surgical excision, and earlier mortality. As EMP2 expression was independent of other known biomarkers such as the estrogen receptor and progesterone receptor, EMP2 represents a unique biomarker for patients who are not responsive to current hormone or chemotherapy. Wadehra et al. (2006) Cancer 107:90-8. Moreover, EMP2 expression level positively correlates with the increasing pre-malignant potential of proliferative endometrium. In other words, there is a gradation of endometrial EMP2 expression, with minimal expression in normal proliferative or quiescent premenopausal endometrium, and increasing expression in patients with disordered proliferative endometrium, endometrial hyperplasia, and endometrium carcinomas.

In the endometrium, EMP2 expression is regulated by progesterone and required for successful blastocyst implantation. Wadehra et al. (2006) Dev Biol 292:430-41; Wadehra et al. (2008) Reprod Biol Endocrinol 6:15. EMP2 appears to regulate trafficking of various proteins and glycolipids by facilitating transfer of molecules from post-Golgi endosomal compartments to appropriate plasma membrane locations. Specifically, EMP2 is thought to facilitate the appropriate trafficking of select molecules into glycolipids-enriched lipid raft microdomains (GEMs). Wadehra et al. (2004) Mol Biol Cell 15:2073-83. GEMs are cholesterol rich microdomains which are often associated with chaperones, receptosomes, and protein complexes that are important for efficient signal transduction. Leitinger et al. (2002) J Cell Sci 115:963-72; Moffett et al. (2000) J Biol Chem 275:2191-8. Moreover, GEMs are involved in correct sorting of proteins from the Golgi apparatus to plasma membrane. Abrami et al. (2001) J Biol Chem 276:30729-36; Galbiati et al. (2001) Cell 106:403-11; Gruenberg et al. (1995) Curr Opin Cell Biol 7: 552-63. In this respect, modulation of EMP2 expression levels or its location on the plasma membrane alters the surface repertoire of several classes of molecules including integrins, focal adhesion kinase, class I major histocompatibility molecules and other immunoglobulin super-family members such as CD54 and GPI-linked proteins. Wadehra et al. (2005) Dev Biol 287:336-45; Wadehra et al. (2003) Clinical Immunology 107:129-36; Morales et al. (2008) Invest Opthalmol Vis Sci 50(1):462-9. Furthermore, concordant with the role of tetraspans, EMP2 is thought to curate molecules on the plasma membrane to regulate the activity of specific signaling complexes.

Accordingly, there is a need for new compositions and methods to treat and prevent cancers, to treat and prevent angiogenesis-related diseases and disorders, and to reduce neovascularization (i.e., the proliferation of blood vessels in tissue not normally containing them). Disclosed herein are methods and composition for reducing neovascularization by targeting EMP2. Disclosed herein are also methods and compositions for treating and preventing uterine cancer. Specifically disclosed herein are methods and compositions for treating and preventing endometrial cancer.

### BRIEF SUMMARY OF THE INVENTION

In one embodiment, the invention relates to a method of treating a patient for uterine cancer. In a specific embodiment, the method comprises co-administering to the patient an effective amount of an anti-angiogenic agent or a chemotherapeutic agent and an effective amount of an anti-EMP2 antibody to a subject in need thereof. In a specific embodiment, the anti-EMP2 antibody specifically binds to an epitope in the second extracellular loop of EMP2. In a specific embodiment, the epitope in the second extracellular loop of EMP2 comprises the amino acid sequence DIHDKNAKFYPVTREGSYG.

In certain embodiments, the anti-EMP2 antibody is a humanized or fully human antibody. In certain embodiments, the anti-EMP2 antibody is a diabody. In certain embodiments, the anti-EMP2 antibody is a triabody. In certain embodiments, the anti-EMP2 antibody is a minibody. In certain embodiments, the anti-EMP2 antibody is a single chain antibody.

In certain embodiments, the effective amount of an anti-angiogenic agent or a chemotherapeutic agent and an effective amount of an anti-EMP2 antibody further comprises a physiological acceptable carrier or a pharmaceutically acceptable carrier.

In certain embodiments, the anti-EMP2 antibody competes with an antibody that comprises the heavy and light chain variable regions of a KS49, a KS41, a KS83, or a KS89 diabody. In certain embodiments, the antibody shares 90% amino acid identity with heavy and light chain variable regions of a KS49, a KS41, a KS83, or a KS89 diabody.

In certain embodiments, the anti-angiogenic agent is a VEGF inhibitor. In a specific embodiment, the VEGF inhibitor is an anti-VEGF antibody. In a specific embodiment, the anti-VEGF antibody is bevacizumab. In a specific embodiment, the anti-VEGF antibody is pazopanib, sorafenib, sunitinib, vandeteanib, cabozantinib, ponatinib, axitinib, or aflibercept.

In certain embodiments, the chemotherapeutic agent is a DNA damaging chemotherapeutic agent. In a specific embodiment, the DNA damaging chemotherapeutic agent is a topoisomerase I inhibitor, topoisomerase II inhibitor, alkylating agent, DNA intercalator, free radical generator, or nucleoside mimetic. In a specific embodiment, the topoisomerase I inhibitor is irinotecan, topotecan, camptothecin and analogs or metabolites thereof or doxorubicin. In specific embodiments, the topoisomerase II inhibitor is etoposide, teniposide, or daunorubicin. In specific embodiments, the alkylating agent is melphalan, chlorambucil, busulfan, thiotepa, ifosfamide, carmustine, lomustine, semustine, streptozocin, decarbazine, methotrexate, mitomycin C, or cyclophosphamide. In specific embodiments, the DNA intercalator is cisplatin, oxaliplatin, or carboplatin. In specific embodiments, the free radical generator is bleomycin. In specific embodiments, the nucleoside mimetics is 5-fluorouracil, capecitibine, gemcitabine, fludarabine, cytarabine, mercaptopurine, thioguanine, pentostatin, or hydroxyurea.

In certain embodiments, the chemotherapeutic agent is a cell replication disrupting agent. In specific embodiments, the cell replication disrupting agent is paclitaxel, docetaxel, vincristine, vinblastin, thalidomide, lenalidomide, CC-5013, CC-4047, protein tyrosine kinase inhibitors, proteasome inhibitors, NF-κB inhibitors, or related analogs thereof. In specific embodiments, the protein tyrosine kinase inhibitor is imatinib mesylate or gefitinib. In a specific embodiment, the proteasome inhibitor is bortezomib. In a specific embodiment, the NF-κB inhibitor is an inhibitor of IκB kinase. In a specific embodiment, the cell replication disrupting agent is an antibody.

In certain embodiments, the uterine cancer is endometrial cancer.

In certain embodiments, the method further comprises administering to the patient an effective amount of at least one additional anti-cancer agent. In certain embodiments, the at least one additional anti-cancer agent is selected from the group consisting of platinum-based chemotherapy drugs, taxanes, tyrosine kinase inhibitors, anti-EGFR antibodies, anti-ErbB2 antibodies, and combinations thereof.

In certain embodiments, the at least one additional anti-cancer agent is an EGFR inhibitor. In certain embodiments, the EGFR inhibitor is an anti-EGFR antibody. In certain embodiments, the anti-EGFR antibody is cetuximab. In certain embodiments, the anti-EGFR antibody is matuzumab, panitumumab, or nimotuzumab. In certain embodiments, the EGFR inhibitor is a small molecule inhibitor of EGFR signaling. In certain embodiments, the small molecule inhibitor of EGFR signaling is gefitinib, lapatinib, canertinib, pelitinib, erlotinib HCL, PKI-166, PD158780, or AG 1478.

In certain embodiments, the anti-EMP2 antibody is conjugated with an effector moiety. In certain embodiments, the effector moiety is a toxic agent. In certain embodiments, the toxic agent is such as ricin.

In certain embodiments, the anti-EMP2 and the anti-angiogenic agents are administered simultaneously. In certain embodiments, the anti-EMP2 and the anti-angiogenic agents are administered sequentially. In certain embodiments, the anti-EMP2 and the chemotherapeutic agents are administered simultaneously. In certain embodiments, the anti-EMP2 and the chemotherapeutic agent are administered sequentially.

In certain embodiments, the anti-EMP2 antibodies are used in vaccine therapies for the cancer.

In certain embodiments, the patient is a human or a mammal.

In certain embodiments, the method further comprises a companion diagnostic. In certain embodiments, the companion diagnostic comprises an anti-EMP2 antibody. In certain embodiments, the anti-EMP2 antibody is conjugated to a diagnostic moiety.

In another embodiment, this invention relates to a method of treating a non-neoplastic condition, comprising administering an effective amount of an EMP2 inhibitor to a subject in need thereof. In certain embodiments, the anti-EMP2 antibody specifically binds to an epitope in the second extracellular loop of EMP2. In certain embodiments, the epitope in the second extracellular loop of EMP2 comprises the amino acid sequence DIHDKNAKFYPVTREGSYG.

In certain embodiments, the anti-EMP2 antibody is a humanized or fully human antibody. In certain embodiments, the anti-EMP2 antibody is a diabody. In certain embodiments, the anti-EMP2 antibody is a triabody. In certain embodiments, the anti-EMP2 antibody is a minibody. In certain embodiments, the anti-EMP2 antibody is a single chain antibody.

In certain embodiments, the effective amount of an anti-EMP2 antibody further comprises a physiological acceptable carrier or a pharmaceutically acceptable carrier.

In certain embodiments, the anti-EMP2 antibody competes with an antibody comprising the heavy and light chain variable regions of a KS49, a KS41, a KS83, or a KS89 diabody. In certain embodiments, the antibody shares 90% amino acid identity with heavy and light chain variable regions of a KS49, a KS41, a KS83, or a KS89 diabody.

In certain embodiments, the EMP2 inhibitor is a shRNA, a ribozyme, or an anti-EMP2 antibody.

In certain embodiments, the EMP2 inhibitor is administered before, after or concomitantly with an anti-angiogenic agent. In certain embodiments, the anti-angiogenic agent is a VEGF inhibitor. In certain embodiments, the VEGF inhibitor comprises an anti-VEGF antibody. In certain embodiments, the anti-VEGF antibody is bevacizumab.

In certain embodiments, the anti-VEGF antibody is pazopanib, sorafenib, sunitinib, vandeteanib, cabozantinib, ponatinib, axitinib, or aflibercept.

In certain embodiments, the EMP2 inhibitor is administered before, after or concomitantly with a chemotherapeutic agent.

In certain embodiments, the chemotherapeutic agent is a DNA damaging chemotherapeutic agent or a replication disrupting agent. In certain embodiments, the method further comprises administering to the patient an effective amount of at least one additional anti-cancer agent. In certain embodiments, the at least one additional anti-cancer agent is selected from the group consisting of platinum-based chemotherapy drugs, taxanes, tyrosine kinase inhibitors, anti-EGFR antibodies, anti-ErbB2 antibodies, and combinations thereof.

In certain embodiments, the at least one additional anti-cancer agent comprises an EGFR inhibitor. In certain embodiments, the EGFR inhibitor comprises an anti-EGFR antibody. In certain embodiments, the anti-EGFR antibody comprises cetuximab. In certain embodiments, the anti-EGFR antibody is matuzumab, panitumumab, or nimotuzumab. In certain embodiments, the EGFR inhibitor is a small molecule inhibitor of EGFR signaling. In certain embodiments, the small molecule inhibitor of EGFR signaling is gefitinib, lapatinib, canertinib, pelitinib, erlotinib HCL, PKI-166, PD158780, or AG 1478.

In certain embodiments, the anti-EMP2 antibody is conjugated with an effector moiety. In certain embodiments, the effector moiety is a toxic agent. In certain embodiments, the toxic agent is such as ricin.

In certain embodiments, the anti-EMP2 antibodies are used in vaccine therapies for the cancer.

In certain embodiments, the patient is a human or a mammal.

In certain embodiments, the method further comprises a companion diagnostic. In certain embodiments, the companion diagnostic comprises an anti-EMP2 antibody. In certain embodiments, the anti-EMP2 antibody is conjugated to a diagnostic moiety.

In certain embodiments, the non-neoplastic condition comprises rheumatoid arthritis, psoriasis, atherosclerosis, diabetic retinopathy, retrolentral fibroplasia, thyroid hyperplasia, chronic inflammation, lung inflammation, nephrotic syndrome, preclampsia, ascites, pericardial effusion, or pleural effusion.

In another embodiment, the invention relates to a method of treating a condition characterized by neovascularization. In certain embodiments, the method comprises administering an effective amount of an EMP2 inhibitor to a subject in need thereof.

In another embodiment, the EMP2 inhibitor is a shRNA. In one embodiment, the EMP2 inhibitor is a ribozyme. In one embodiment, the EMP2 inhibitor is an anti-EMP2 antibody. In a specific embodiment, the anti-EMP2 antibody is a human antibody.

In another embodiment, the condition characterized by neovascularization is a neoplastic condition. In a specific embodiment, the neoplastic condition is breast, lung, esophagus, gastric, colon, rectum, liver, ovary, cervix, prostate, pancreas, or renal carcinoma. In certain embodiments, the neoplastic condition is the coma, arrhenoblastoma, fibrosarcoma, choriocarcinoma, head cancer, neck cancer, nasopharyngeal carcinoma, laryngeal carcinoma, hepatoblastoma, Karposi's sarcoma, melanoma, skin carcinoma, hemangioma, hemangioblastoma, retinoblastoma, astrocystoma, glioblastoma, Schwannoma, oligodendroglioma, medulloblastoma, neuroblastoma, rhabdomyosarcoma, osteogenic sarcoma, leiomyosarcomas, urinary tract carcinoma, thyroid carcinoma, Wilm's tumor, abnormal vascular proliferation associated with phakomatoses, and Meigs' syndrome.

In another embodiment, the condition characterized by neovascularization is a non-neoplastic condition. In specific embodiments, the non-neoplastic condition is rheumatoid arthritis, psoriasis, atherosclerosis, diabetic retinopathy, retrolentral fibroplasia, thyroid hyperplasia, chronic inflammation, lung inflammation, nephrotic syndrome, preclampsia, ascites, pericardial effusion, or pleural effusion.

In one embodiment the EMP2 inhibitor is administered before, after or concomitantly with a VEGF inhibitor. In one embodiment, the EMP2 inhibitor is administered concurrently with an anti-angiogenic or chemotherapeutic agent described herein.

### BRIEF DESCRIPTION OF THE DRAWINGS

FIG. 1 depicts that EMP2 IgG1 treatment reduces tumor load. (A) HEC1a/EMP2 cells were injected s.c. into nude Balb/c female mice. When tumors reached 4 mm², control of EMP2 IgG1 antibodies were systemically injected at 10 mg/kg. Mice were treated weekly, and tumor volume was monitored using calipers. n=6. *, p<0.05. (B) Kaplan-Meier survival analysis of the outcome of athymic mice treated with anti-EMP2 IgG1 or a control antibody. N=6; p=0.02. (C) Treated tumors were harvested and fixed. Tumors were visualized using hematoxylin and eosin. Magnification: 4×.

FIG. 2 depicts that EMP2 expression increases tumor vasculature. (A) 1×10⁶ HEC1a/EMP2, HEC1a/V, or HEC1a/RIBO cells were injected into Balb/c nude mice. After ays, tumors were harvested, fixed, and stained with Masson's trichrome. N=6. (B) Tumors were stained using Lycopersicon esculentum lectin and DAPI. (C) Tumors were stained for CD34 expression. In all images, arrows highlight tumor vasculature. Magnification: 20×. Scale bar=100 μM. (D) The numbers of CD34-positive blood vessels in 6 high power fields (×200) from at least 2 independent tumors were counted and averaged. Bars, mean±SE.

FIG. 3 depicts that EMP2 promotes angiogenesis. (A) Chemotactic effects on the migration of HUVECs were measured using a standard Boyden chamber assay. Experiments were repeated three times with data representing the mean±SE. (B) HUVEC cells migration was measured using a “scratch” wound healing assay. Endothelial cells were cultured in tumor cell supernatant, and wound closure was measured using microscopy. (C) HUVEC cells were plated on low growth factor matrigel in the presence of cultured media from HEC1a/EMP2, HEC1a/V, or HEC1a/RIBO cells. The experiment was repeated at least three times, and a representative image is shown. Capillary like tube formation was quantitated by measuring the number of tubes (D) as well as the tube diameter (E). The data in the graph is the mean±SEM of three fields using three independent experiments.

FIG. 4 depicts that EMP2 regulates VEGF expression. (A) A Boyden chamber assay was used to determine human aortic endothelial cell (HAEC) response to cultured tumor cell supernatants. In some experiments, the antiVEGF antibody Avastin® which binds soluble VEGF was added at 10μg/ml to the cultured supernatant. (B) The expression of total VEGF was measured using Western blot analysis. EMP2 expression was visualized to verify modulation of EMP2 in the cell lines; β-Actin was used as the loading control. (C) Secreted VEGF was measured using an ELISA. (D) Semi-quantitative expression of VEGF mRNA was determined using RTPCR. The experiment was repeated three times with similar results, and a representative blot is shown. GAPDH expression serves as a loading control. (E) Left, To determine the mechanism for VEGF regulation, HIF-1 a and PP ARy expression was determined using Western blot analysis. β-Actin expression serves as the loading control. Right, Quantization of HIF-1a expression relative to β-Actin for three independent experiments. The data represents the mean±SEM. (F) EMP2 expression was downregulated using either a ribozyme (HEC1a/RIBO) or shRNA lentiviral constructs (HEC1a/shRNA). Appropriate vehicle control cells are included. Western blot analysis was used to show the expression of EMP2, HIF-1a, and β-Actin in these cells.

FIG. 5 depicts that EMP2 regulates VEGF through the FAK-Src pathway. (A) Exogenous VEGF was added to HEC1a/RIBO or HEC1a/shRNA cells at 20 or 50 g/ml. Capillary like tube formation was measured using phase contrast microscopy after 12-24 hrs. The experiment was repeated three times, with the data presented as the mean±SEM. (B) HEC-1A/EMP2 cells were incubated with the PP2, PP3, Dasatinib, Erlotinib, Ly294002, AKTi VIII, a DMSO vehicle control, or a media control for 24 hrs in a 1.0% hypoxic chamber. Cells were probed for the protein expression of HIF-1a, p-FAK, p-SRC, p-AKT, and β-Actin.

FIG. 6 depicts EMP2 IgG1 treatment alters the tumor microenvironment. (A) An equivalent number of HEC1a/EMP2 endometrial tumor cells were treated for 12 hours with varying concentrations of EMP2 IgG 1 or control IgG. Supernatants were collected and added to HUVEC plated on low growth factor matrigel. Capillary tube formation was measured using phase contrast microscopy after 12-24 hours. The experiment was repeated three times, with the data presented as the mean±SEM. (B) neovascularization of HEC1a/EMP2 tumors treated with EMP2 IgG1 or control antibodies was visualized using Lycopersicon esculentum lectin and DAPI in surviving tumor clusters. (C) Mason's trichtome stain was used to identify basement membrane sleeves in central areas of the tumor. N=3, with a representative image shown. Magnification, 10×.

FIG. 7 depicts the sequences of the heavy and light chain variable regions of anti-EMP2 antibodies.

### DETAILED DESCRIPTION OF THE INVENTION

### Introduction

Uterine cancer is the fourth most common type of cancer found in women. The most common type of uterine cancer is endometrial cancer. Endometrial cancer originates in cells lining the uterus. In 2012, it was estimated that there were about 47,130 new cases of endometrial cancer diagnosed each year in the United States, resulting in 8,010 deaths annually. See National Cancer Institute website on Endometrial Cancer. The disease most typically occurs in postmenopausal women, with the average age at the time of diagnosis being about 60 years. Often, the development of endometrial cancer is preceded by premalignant neoplastic growth of endometrial glandular cells, a condition that is usually apparent upon histological examination as atypical hyperplasia, or Endometrial Intraepithelial Neoplasia (EIN). Hecht et al. (2005) Mod. Pathol 18:324-330; Mutter et al. (2000) J. Pathol 190:462-469. However, by the time that EIN is detected, cancer is already present in approximately 39% of women. Baak, et al. (2005) J. Clin. Pathol. 58:1-6.

EMP2 (SEQ ID NO:1) is a novel oncogene upregulated in a number of cancers in women. In endometrial cancer, for example, EMP2 expression promotes endometrial cancer growth in vivo, and to date, it is the only biomarker identified to predict endometrial cancer prognosis and survival. Given its expression profile and importance in disease pathogenesis, a recombinant antibody fragment (diabody) to EMP2 has been generated and shown it induce necrosis in vivo.

(EMP2; ACCESSION P54851)
SEQ ID NO: 1
MLVLLAFIIA FHITSAALLF IATVDNAWWV GDEFFADVWR
ICTNNTNCTV INDSFQEYST LQAVQATMIL STILCCIAFF
IFVLQLFRLK QGERFVLTSI IQLMSCLCVM IAASIYTDRR
EDIHDKNAKF YPVTREGSYG YSYILAWVAF ACTFISGMMY
LILRKRK

Using endometrial cancer cells, anti-EMP2 IgG 1 treatment significantly reduced tumor load with a net improvement in survival. Within tumors, significant necrosis was observed. Also, expression of ribozymes or specific shRNA constructs that reduced EMP2 revealed a similar histology. In particular, severe necrosis was observed in tumors with reduced EMP2. Although we had previously observed increased cell death in vitro after exposure to antibodies that recognize EMP2, the necrotic response and the studies reported in the instant disclosure link EMP2 with control of VEGF expression through HIF-1a. Notably, HEC-1A cells that were genetically modified for EMP2 expression showed a positive correlation between EMP2 levels and tumor vascularity. Cell supernatants from cells that were genetically modified for EMP2 expression showed a positive correlation between EMP2 levels, and endothelial cell migration and tube formation of two independent endothelial types. Levels of VEGF and Hif-1a were concordant also with expression levels of EMP2, and blockade of EMP2 using an anti-EMP2 antibody showed a dose dependent decrease in vascularization. Additional investigations will help identify how EMP2 controls the hypoxic response. All of the studies presented here point to a significant effect on HIF-1a and VEGF through control of EMP2 expression and suggest the mechanism for the clinical association of high EMP2 expression with aggressive, more advanced tumors.

Several oncogenes stimulate angiogenesis. Without being bound by theory it is postulated that EMP2 activates HIF-1a through a Src dependent mechanism. This stimulation appears to be independent of AKT activation suggesting that EMP2 and AKT function independently of one another. Moreover, although AKT inhibitors did not suppress EMP2 mediated HIF-1a activation, erlotinib and Ly294002, the PI3 kinase inhibitor, are sufficient to inhibit its expression.

### Antibodies

The present invention provides anti-EMP2 antibodies, generally therapeutic and/or diagnostic antibodies as described herein. Antibodies that find use in the present invention can take on a number of formats as described herein, including traditional antibodies as well as antibody derivatives, fragments and mimetics, described below. Essentially, the invention provides antibody structures that contain a set of 6 CDRs as defined herein (including small numbers of amino acid changes as described below). The anti-EMP2 CDRs are provided in U.S. Pat. No. 8,318,906, incorporated by reference in its entirety.

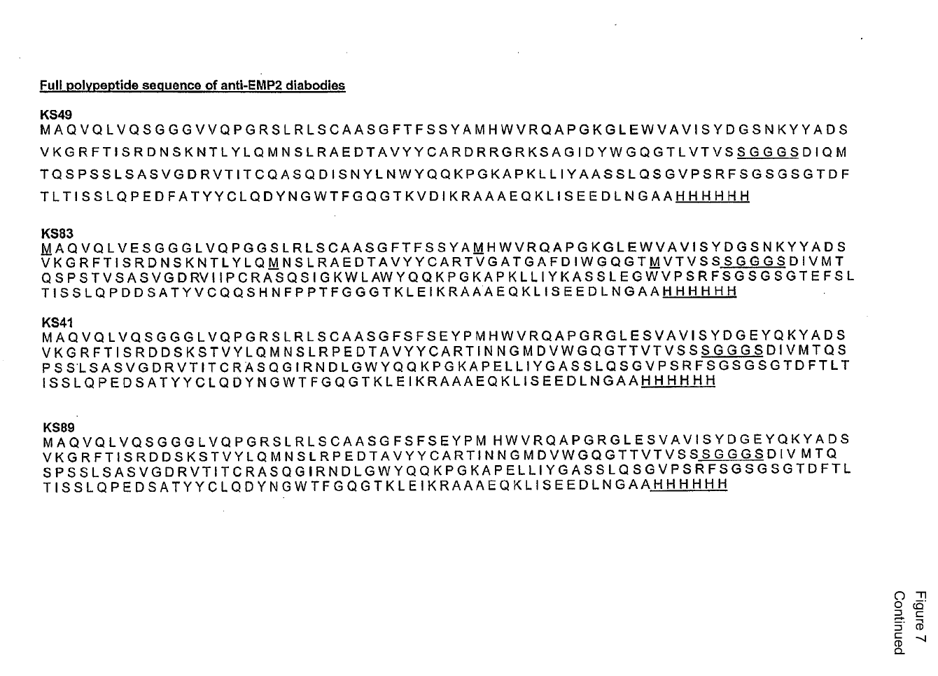

The heavy and light chain variable regions sequences of anti-EMP2 antibodies are provided in FIG. 7.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Thus, “isotype” as used herein is meant any of the subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE. It should be understood that therapeutic antibodies can also comprise hybrids of isotypes and/or subclasses.

The amino-terminal portion of each chain includes a variable region of about 100 to 110 or more amino acids primarily responsible for antigen recognition. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5^{th} Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

Throughout the present specification, the Kabat numbering system is generally used when referring to a residue in the variable domain (approximately, residues 1-107 of the light chain variable region and residues 1-113 of the heavy chain variable region) (e.g, Kabat et al., supra (1991)).

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope. For example, as described herein the antibodies bind to an epitope in the presumptive second extracellular domain of EMP2.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

In some embodiments, the epitope is derived from SEQ ID NO:2, wherein SEQ ID NO:2 is EDIHDKNAKFYPVTREGSYG and represents a 20-mer polypeptide sequence from the second extracellular loop of human EMP2

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.”

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5^{th} edition, NIH publication, No. 91-3242, E.A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230.

Of interest in the present invention are the Fc regions. By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

In some embodiments, the antibodies are full length. By “full length antibody” herein is meant the structure that constitutes the natural biological form of an antibody, including variable and constant regions, including one or more modifications as outlined herein.

Alternatively, the antibodies can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively. Structures that still rely

In one embodiment, the antibody is an antibody fragment. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546, entirely incorporated by reference) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (viii) bispecific single chain Fv (WO 03/11161, hereby incorporated by reference) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference). In some embodiments, the antibody can be a mixture from different species, e.g. a chimeric antibody and/or a humanized antibody. That is, in the present invention, the CDR sets can be used with framework and constant regions other than those specifically described by sequence herein.

In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213, all entirely incorporated by reference). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference. In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

In one embodiment, the antibodies of the invention can be multispecific antibodies, and notably bispecific antibodies. These are antibodies that bind to two (or more) different antigens, or different epitopes on the same antigen.

In some embodiments the antibodies are diabodies.

In one embodiment, the antibody is a minibody. Minibodies are minimized antibody-like proteins comprising a scFv joined to a CH3 domain. Hu et al., 1996, Cancer Res. 56:3055-3061, entirely incorporated by reference. In some cases, the scFv can be joined to the Fc region, and may include some or the entire hinge region.

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. For instance, an isolated antibody that specifically binds to EMP2 is substantially free of antibodies that specifically bind antigens other than EMP2.

An isolated antibody that specifically binds to an epitope, isoform or variant of human EMP2 or murine EMP2 may, however, have cross-reactivity to other related antigens, for instance from other species, such as EMP2 species homologs. Moreover, an isolated antibody may be substantially free of other cellular material and/or chemicals.

Isolated monoclonal antibodies, having different specificities, can be combined in a well defined composition. Thus, for example all possible combinations of the disclosed antibodies can be combined in a single formulation, if desired.

For example, as described in U.S. Pat. No. 8,318,906, the following human-origin antibody sequences encode for high-avidity antibodies specific for human (KS49, KS83) and mouse (KS83) EMP2 and have antibody variable region heavy and light chains suitable for use in either aspect of the invention:

KS49 heavy chain-
M A Q V Q L V Q S G G G V V Q P G R S L R L S C
A A S G F T F S S Y A M H W V R Q A P G K G L E
W V A V I S Y D G S N K Y Y A D S V K G R F T I
S R D N S K N T L Y L Q M N S L R A E D T A V Y
Y C A R D R R G R K S A G I D Y W G Q G T L V T
V S S
KS49 light chain-
D I Q M T Q S P S S L S A S V G D R V T I T C Q
A S Q D I S N Y L N W Y Q Q K P G K A P K L L I
Y A A S S L Q S G V P S R F S G S G S G T D F T
L T I S S L Q P E D F A T Y Y C L Q D Y N G W T
F G Q G T K V D I K R A A A E Q K L I S E E D L
N G A A
KS83 heavy chain-
M A Q V Q L V E S G G G L V Q P G G S L R L S C
A A S G F T F S S Y A M H W V R Q A P G K G L E
W V A V I S Y D G S N K Y Y A D S V K G R F T I
S R D N S K N T L Y L Q M N S L R A E D T A V Y
Y C A R T V G A T G A F D I W G Q G T M V T V S
S S
KS83 light chain-
D I V M T Q S P S T V S A S V G D R V I I P C R
A S Q S I G K W L A W Y Q Q K P G K A P K L L I
Y K A S S L E G W V P S R F S G S G S G T E F S
L T I S S L Q P D D S A T Y V C Q Q S H N F P P
T F G G G T K L E I K R A A A E Q K L I S E E D
L N G A A
Other diabodies for use according to either
aspect of the invention include KS41 and KS89:
KS41 Heavy Chain-
M A Q V Q L V Q S G G G L V Q P G R S L R L S C
A A S G F S F S E Y P M H W V R Q A P G R G L E
S V A V I S Y D G E Y Q K Y A D S V K G R F T I
S R D D S K S T V Y L Q M N S L R P E D T A V Y
Y C A R T I N N G M D V W G Q G T T V T V S S
KS41 Light Chain-
D I V M T Q S P S S L S A S V G D R V T I T C R
A S Q G I R N D L G W Y Q Q K P G K A P E L L I
Y G A S S L Q S G V P S R F S G S G S G T D F T
L T I S S L Q P E D S A T Y Y C L Q D Y N G W T
F G Q G T K L E I K R A A A E Q K L I S E E D L
N G A A
KS89 Heavy Chain-
M A Q V Q L V Q S G G G L V Q P G R S L R L S C
A A S G F S F S E Y P M H W V R Q A P G R G L E
S V A V I S Y D G E Y Q K Y A D S V K G R F T I
S R D D S K S T V Y L Q M N S L R P E D T A V Y
Y C A R T I N N G M D V W G Q G T T V T V S S
KS89 Light Chain-
D I V M T Q S P S S L S A S V G D R V T I T C R
A S Q G I R N D L G W Y Q Q K P G K A P E L L I
Y G A S S L Q S G V P S R F S G S G S G T D F T
L T I S S L Q P E D S A T Y Y C L Q D Y N G W T
F G Q G T K L E I K R A A A E Q K L I S E E D L
N G A A

Anti-EMP-2 variable region sequences, used to encode proteins on backbones including for native antibody, fragment antibody, or synthetic backbones, can avidly bind EMP-2. Via this binding, these proteins can be used for EMP-2 detection, and to block EMP-2 function. Expression of these variable region sequences on native antibody backbones, or as an scFv, triabody, diabody or minibody, labeled with radionuclide, are particularly useful in the in vivo detection of EMP-2 bearing cells. Expression on these backbones or native antibody backbone are favorable for blocking the function of EMP-2 and/or killing EMP-2 bearing cells (e.g. gynecologic tumors) in vivo.

In some embodiments, the present invention provides anti-EMP-2 sequences comprising CDR regions of an antibody selected from KS49, KS83, KS41, and KS89. The CDR regions provided by the invention may be used to construct an anti-EMP-2 binding protein, including without limitation, an antibody, a scFv, a triabody, a diabody, a minibody, and the like. In a certain embodiment, an anti-EMP-2 binding protein of the invention will comprise at least one CDR region from an antibody selected from KS49, KS83, KS41, and KS89. Anti-EMP-2 binding proteins may comprise, for example, a CDR-H1, a CDR-H2, a CDR-H3, a CDR-L1, a CDR-L2, a CDR-L3, or combinations thereof, from an antibody provided herein. In particular embodiments of the invention, an anti-EMP-2 binding protein may comprise all three CDR-H sequences of an antibody provided herein, all three CDR-L sequences of an antibody provided herein, or both. Anti-EMP2 CDR sequences may be used on an antibody backbone, or fragment thereof, and likewise may include humanized antibodies, or antibodies containing humanized sequences. These antibodies may be used, for example, to detect EMP-2, to detect cells expressing EMP-2 in vivo, or to block EMP-2 function. In some embodiments, the CDR regions may be defined using the Kabat definition, the Chothia definition, the AbM definition, the contact definition, or any other suitable CDR numbering system.

In some embodiments, the CDRs are as follows:

CDR 1 Heavy
(SEQ ID NO.: 14)
SYAMH (49)
(SEQ ID NO.: 14)
SYAMH (83)
(SEQ ID NO.: 15)
EYPMH (41)
(SEQ ID NO.: 15)
EYPMH (89)
CDR 2 Heavy
(SEQ ID NO.: 16)
VISYDGSNKYYADSVKG (49)
(SEQ ID NO.: 16)
VISYDGSNKYYADSVKG (83)
(SEQ ID NO.: 17)
VISYDGEYQKYADSVKG (41)
(SEQ ID NO.: 17)
VISYDGEYQKYADSVKG (89)
CDR 3 Heavy
(SEQ ID NO.: 39)
DRRGRKSAGIDY (49)
(SEQ ID NO.: 37)
TVGATGAFDI (83)
(SEQ ID NO.: 41)
TINNGMDV (41)
(SEQ ID NO.: 41)
TINNGMDV (89)
CDR 1 Light
(SEQ ID NO.: 19)
QASQDISNYLN (49)
(SEQ ID NO.: 18)
RASQSIGKWLA (83)
(SEQ ID NO.: 20)
RASQGIRNDLG (41)
(SEQ ID NO.: 20)
RASQGIRNDLG (89)

In some embodiments, the CDRs are as follows:

Diabody sequence (KS49)
Heavy chain, KS49
M A Q V Q L V Q S G G G V V Q P G R S L R L S C
A A S G F T F S S Y A M H W V R Q A P G K G L E
W V A V I S Y D G S N K Y Y A D S V K G R F T I
S R D N S K N T L Y L Q M N S L R A E D T A V Y
Y C A R D R R G R K S A G I D Y W G Q G T L V T
V S
CDR1
SYAMH
CDR2
VISYDGSNKYYADSVKG
Light chain, KS49
D I Q M T Q S P S S L S A S V G D R V T I T C Q
A S Q D I S N Y L N W Y Q Q K P G K A P K L L I
Y A A S S L Q S G V P S R F S G S G S G T D F T
L T I S S L Q P E D F A T Y Y C L Q D Y N G W T
F G Q G T K V D I K R A A A E Q K L I S E E D L
N G A A
CDR1
QASQDISNYLN
CDR2
AASSLQS
Diabody sequence (KS83)
Heavy chain, KS83
M A Q V Q L V E S G G G L V Q P G G S L R L S C
A A S G F T F S S Y A M H W V R Q A P G K G L E
W V A V I S Y D G S N K Y Y A D S V K G R F T I
S R D N S K N T L Y L Q M N S L R A E D T A V Y
Y C A R T V G A T G A F D I W G Q G T M V T V S
S
CDR1
SYAMH
CDR2
VISYDGSNKYYADSVKG
Light Chain, KS83
D I V M T Q S P S T V S A S V G D R V I I P C R
A S Q S I G K W L A W Y Q Q K P G K A P K L L I
Y K A S S L E G W V P S R F S G S G S G T E F S
L T I S S L Q P D D S A T Y V C Q Q S H N F P P
T F G G G T K L E I K R A A A E Q K L I S E E D
L N G A A
CDR1
RASQSIGKWLA
CDR2
KASSLEG
Diabody (KS41)
Heavy Chain, KS41
M A Q V Q L V Q S G G G L V Q P G R S L R L S C
A A S G F S F S E Y P M H W V R Q A P G R G L E
S V A V I S Y D G E Y Q K Y A D S V K G R F T I
S R D D S K S T V Y L Q M N S L R P E D T A V Y
Y C A R T I N N G M D V W G Q G T T V T V S S
CDR1
EYPMH
CDR2
VISYDGEYQKYADSVKG
Light Chain, KS41
D I V M T Q S P S S L S A S V G D R V T I T C R
A S Q G I R N D L G W Y Q Q K P G K A P E L L I
Y G A S S L Q S G V P S R F S G S G S G T D F T
L T I S S L Q P E D S A T Y Y C L Q D Y N G W T
F G Q G T K L E I K R A A A E Q K L I S E E D L
N G A A
CDR1
RASQGIRNDLG
CDR2
GASSLQS
Diabody sequence (KS89)
Heavy Chain, KS89
M A Q V Q L V Q S G G G L V Q P G R S L R L S C
A A S G F S F S E Y P Mt H W V R Q A P G R G L
E S V A V I S Y D G E Y Q K Y A D S V K G R F T
I S R D D S K S T V Y L Q M N S L R P E D T A V
Y Y C A R T I N N G M D V W G Q G T T V T V S S
CDR1
EYPMH
CDR2
VISYDGEYQKYADSVKG
Light Chain, KS89
D I V Met T Q S P S S L S A S V G D R V T I T C
R A S Q G I R N D L G W Y Q Q K P G K A P E L L
I Y G A S S L Q S G V P S R F S G S G S G T D F
T L T I S S L Q P E D S A T Y Y C L Q D Y N G W
T F G Q G T K L E I K R A A A E Q K L I S E E D
L N G A A
CDR1
RASQGIRNDLG
CDR2
GASSLQS

In some embodiments, the invention provides antibodies (e.g., diabodies, minibodies, triabodies) or fragments thereof having the CDRs of a diabody selected from KS49, KS83, KS41, and KS89. In some embodiments these antibodies lack the polyhistine tag. In other embodiments, the diabodies possess the light and heavy chain of a KS49, KS83, KS41, or KS89 diabody. In still other embodiments, the antibodies are substantially identical in sequence to a diabody selected from the group consisting of KS49, KS83, KS41, and KS89 with or without the polyhistidine tag. In still other embodiments, the antibodies are substantially identical in sequence to the light and heavy chain sequences of a diabody selected from the group consisting of KS49, KS83, KS41, and KS89. These identities can be 65%, 70%, 75%, 80%, 85%, 90%, and preferably 91%, 92%, 93%, 94%, 95%, 96%, 97%, 98% or 99% or greater amino acid sequence identity. In some further embodiments of any of the above, the antibodies comprise CDRs sequences identical to those of the KS49, KS83, KS41, or KS89 diabody.

The anti-EMP2 antibodies of the present invention specifically bind EMP2 ligands (e.g. the human and murine EMP2 proteins of SEQ ID NOs:1 and 2.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

The anti-EMP2 antibodies of the present invention specifically bind EMP2 ligands (e.g. the human and murine EMP2 proteins of SEQ ID NOS:1 and 2. “Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M , at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

Antibody Modifications

The present invention further provides variant antibodies. That is, there are a number of modifications that can be made to the antibodies of the invention, including, but not limited to, amino acid modifications in the CDRs (affinity maturation), amino acid modifications in the Fc region, glycosylation variants, covalent modifications of other types, etc.

By “variant” herein is meant a polypeptide sequence that differs from that of a parent polypeptide by virtue of at least one amino acid modification. Amino acid modifications can include substitutions, insertions and deletions, with the former being preferred in many cases.

In general, variants can include any number of modifications, as long as the function of the protein is still present, as described herein.

However, in general, from 1, 2, 3, 4, 5, 6, 7, 8, 9 or 10 amino acid substitutions are generally utilized as often the goal is to alter function with a minimal number of modifications. In some cases, there are from 1 to 5 modifications, with from 1-2, 1-3 and 1-4 also finding use in many embodiments.

It should be noted that the number of amino acid modifications may be within functional domains: for example, it may be desirable to have from 1-5 modifications in the Fc region of wild-type or engineered proteins, as well as from 1 to 5 modifications in the Fv region, for example. A variant polypeptide sequence will preferably possess at least about 80%, 85%, 90%, 95% or up to 98 or 99% identity to the parent sequences (e.g. the variable regions, the constant regions, and/or the heavy and light chain sequences.) It should be noted that depending on the size of the sequence, the percent identity will depend on the number of amino acids.

By “amino acid substitution” or “substitution” herein is meant the replacement of an amino acid at a particular position in a parent polypeptide sequence with another amino acid. For example, the substitution S100A refers to a variant polypeptide in which the serine at position 100 is replaced with alanine By “amino acid insertion” or “insertion” as used herein is meant the addition of an amino acid at a particular position in a parent polypeptide sequence. By “amino acid deletion” or “deletion” as used herein is meant the removal of an amino acid at a particular position in a parent polypeptide sequence.

By “parent polypeptide”, “parent protein”, “precursor polypeptide”, or “precursor protein” as used herein is meant an unmodified polypeptide that is subsequently modified to generate a variant. Parent polypeptide may refer to the polypeptide itself, compositions that comprise the parent polypeptide, or the amino acid sequence that encodes it. Accordingly, by “parent Fc polypeptide” as used herein is meant an Fc polypeptide that is modified to generate a variant, and by “parent antibody” as used herein is meant an antibody that is modified to generate a variant antibody.

By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been intentionally modified.

By “variant Fc region” herein is meant an Fc sequence that differs from that of a wild-type Fc sequence by virtue of at least one amino acid modification. Fc variant may refer to the Fc polypeptide itself, compositions comprising the Fc variant polypeptide, or the amino acid sequence.

In some embodiments, one or more amino acid modifications are made in one or more of the CDRs of the antibody. In general, only 1 or 2 or 3 amino acids are substituted in any single CDR, and generally no more than from 4, 5, 6, 7, 8 9 or 10 changes are made within a set of CDRs. However, it should be appreciated that any combination of no substitutions, 1, 2 or 3 substitutions in any CDR can be independently and optionally combined with any other substitution.

In some cases, amino acid modifications in the CDRs are referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, although rare, it may be desirable to decrease the affinity of an antibody to its antigen, but this is generally not preferred.

Affinity maturation can be done to increase the binding affinity of the antibody for the antigen by at least about 10% to 50-100-150% or more, or from 1 to 5 fold as compared to the “parent” antibody. Preferred affinity matured antibodies will have nanomolar or even picomolar affinities for the target antigen. Affinity matured antibodies are produced by known procedures. See, for example, Marks et al., 1992, Biotechnology 10:779-783 that describes affinity maturation by variable heavy chain (VH) and variable light chain (VL) domain shuffling. Random mutagenesis of CDR and/or framework residues is described in: Barbas, et al. 1994, Proc. Nat. Acad. Sci, USA 91:3809-3813; Shier et al., 1995, Gene 169:147-155; Yelton et al., 1995, J. Immunol. 155:1994-2004; Jackson et al., 1995, J. Immunol. 154(7):3310-9; and Hawkins et al, 1992, J. Mol. Biol. 226:889-896, for example.

Alternatively, amino acid modifications can be made in one or more of the CDRs of the antibodies of the invention that are “silent”, e.g. that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs of KS49, KS41, KS83, or KS89. In addition, as outlined below, amino acid modifications can also independently and optionally be made in any region outside the CDRs, including framework and constant regions.

In some embodiments, the anti-EMP2 antibodies of the invention are composed of a variant Fc domain. As is known in the art, the Fc region of an antibody interacts with a number of Fc receptors and ligands, imparting an array of important functional capabilities referred to as effector functions. These Fc receptors include, but are not limited to, (in humans) FcγRI (CD64) including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158, correlated to antibody-dependent cell cytotoxicity (ADCC)) and FcγRIIIb (including allotypes FcγRIIIb-NA1 and FcγRIIIb-NA2), FcRn (the neonatal receptor), C1q (complement protein involved in complement dependent cytotoxicity (CDC)) and FcRn (the neonatal receptor involved in serum half-life). Suitable modifications can be made at one or more positions as is generally outlined, for example in U.S. patent application Ser. No. 11/841,654 and references cited therein, US 2004/013210, US 2005/0054832, US 2006/0024298, US 2006/0121032, US 2006/0235208, US 2007/0148170, U.S. Ser. No. 12/341,769, U.S. Pat. No. 6,737,056, U.S. Pat. No. 7,670,600, U.S. Pat. No. 6,086,875 all of which are expressly incorporated by reference in their entirety, and in particular for specific amino acid substitutions that increase binding to Fc receptors.

In addition to the modifications outlined above, other modifications can be made. For example, the molecules may be stabilized by the incorporation of disulphide bridges linking the VH and VL domains (Reiter et al., 1996, Nature Biotech. 14:1239-1245, entirely incorporated by reference). In addition, there are a variety of covalent modifications of antibodies that can be made as outlined below.

Covalent modifications of antibodies are included within the scope of this invention, and are generally, but not always, done post-translationally. For example, several types of covalent modifications of the antibody are introduced into the molecule by reacting specific amino acid residues of the antibody with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues may also be derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole and the like.

In addition, modifications at cysteines are particularly useful in antibody-drug conjugate (ADC) applications, further described below. In some embodiments, the constant region of the antibodies can be engineered to contain one or more cysteines that are particularly “thiol reactive”, so as to allow more specific and controlled placement of the drug moiety. See for example U.S. Pat. No. 7,521,541, incorporated by reference in its entirety herein.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using 125I or 131I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking antibodies to a water-insoluble support matrix or surface for use in a variety of methods, in addition to methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cynomolgusogen bromide-activated carbohydrates and the reactive substrates described in U.S. Pat. Nos. 3,969,287; 3,691,016; 4,195,128; 4,247,642; 4,229,537; and 4,330,440, all entirely incorporated by reference, are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983], entirely incorporated by reference), acetylation of the N-terminal amine, and amidation of any C-terminal carboxyl group.

In addition, as will be appreciated by those in the art, labels (including fluorescent, enzymatic, magnetic, radioactive, etc. can all be added to the antibodies (as well as the other compositions of the invention). Another type of covalent modification is alterations in glycosylation. In another embodiment, the antibodies disclosed herein can be modified to include one or more engineered glycoforms. By “engineered glycoform” as used herein is meant a carbohydrate composition that is covalently attached to the antibody, wherein said carbohydrate composition differs chemically from that of a parent antibody. Engineered glycoforms may be useful for a variety of purposes, including but not limited to enhancing or reducing effector function. A preferred form of engineered glycoform is afucosylation, which has been shown to be correlated to an increase in ADCC function, presumably through tighter binding to the FcγRIIIa receptor. In this context, “afucosylation” means that the majority of the antibody produced in the host cells is substantially devoid of fucose, e.g. 90-95-98% of the generated antibodies do not have appreciable fucose as a component of the carbohydrate moiety of the antibody (generally attached at N297 in the Fc region). Defined functionally, afucosylated antibodies generally exhibit at least a 50% or higher affinity to the FcγRIIIa receptor.

Engineered glycoforms may be generated by a variety of methods known in the art (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473; U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1, all entirely incorporated by reference; (Potelligent® technology [Biowa, Inc., Princeton, N.J.]; GlycoMAb® glycosylation engineering technology [Glycart Biotechnology AG, Zurich, Switzerland]). Many of these techniques are based on controlling the level of fucosylated and/or bisecting oligosaccharides that are covalently attached to the Fc region, for example by expressing an IgG in various organisms or cell lines, engineered or otherwise (for example Lec-13 CHO cells or rat hybridoma YB2/0 cells, by regulating enzymes involved in the glycosylation pathway (for example FUT8 [α1,6-fucosyltranserase] and/or β1-4-N-acetylglucosaminyltransferase III [GnTIII]), or by modifying carbohydrate(s) after the IgG has been expressed. For example, the “sugar engineered antibody” or “SEA technology” of Seattle Genetics functions by adding modified saccharides that inhibit fucosylation during production; see for example 20090317869, hereby incorporated by reference in its entirety. Engineered glycoform typically refers to the different carbohydrate or oligosaccharide; thus an antibody can include an engineered glycoform.

Alternatively, engineered glycoform may refer to the IgG variant that comprises the different carbohydrate or oligosaccharide. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antibody amino acid sequence is preferably altered through changes at the DNA level, particularly by mutating the DNA encoding the target polypeptide at preselected bases such that codons are generated that will translate into the desired amino acids.

Another means of increasing the number of carbohydrate moieties on the antibody is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 and in Aplin and Wriston, 1981, CRC Crit. Rev. Biochem., pp. 259-306, both entirely incorporated by reference.

Removal of carbohydrate moieties present on the starting antibody (e.g. post-translationally) may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131, both entirely incorporated by reference. Enzymatic cleavage of carbohydrate moieties on polypeptides can be achieved by the use of a variety of endo- and exo-glycosidases as described by Thotakura et al., 1987, Meth. Enzymol. 138:350, entirely incorporated by reference. Glycosylation at potential glycosylation sites may be prevented by the use of the compound tunicamycin as described by Duskin et al., 1982, J. Biol. Chem. 257:3105, entirely incorporated by reference. Tunicamycin blocks the formation of protein-N-glycoside linkages.

Another type of covalent modification of the antibody comprises linking the antibody to various nonproteinaceous polymers, including, but not limited to, various polyols such as polyethylene glycol, polypropylene glycol or polyoxyalkylenes, in the manner set forth in, for example, 2005-2006 PEG Catalog from Nektar Therapeutics (available at the Nektar website) U.S. Pat. Nos. 4,640,835; 4,496,689; 4,301,144; 4,670,417; 4,791,192 or 4,179,337, all entirely incorporated by reference. In addition, as is known in the art, amino acid substitutions may be made in various positions within the antibody to facilitate the addition of polymers such as PEG. See for example, U.S. Publication No. 2005/0114037A1, entirely incorporated by reference. The present invention provides a number of antibodies each with a specific set of CDRs (including, as outlined above, some amino acid substitutions). As outlined above, the antibodies can be defined by sets of 6 CDRs, by variable regions, or by full-length heavy and light chains, including the constant regions. In addition, as outlined above, amino acid substitutions may also be made. In general, in the context of changes within CDRs, due to the relatively short length of the CDRs, the amino acid modifications are generally described in terms of the number of amino acid modifications that may be made. While this is also applicable to the discussion of the number of amino acid modifications that can be introduced in variable, constant or full length sequences, in addition to number of changes, it is also appropriate to define these changes in terms of the “% identity”. Thus, as described herein, antibodies included within the invention are 80, 81, 82, 83, 84, 85, 86, 87, 88, 89, 90, 91, 92, 93, 94, 95, 96, 97, 98, 98 or 99% identical to KS49, KS41, KS83, or KS89 described herein.

In some embodiments, antibodies that compete with the antibodies of the invention (for example, with KS49, KS41, KS83, or KS89) for binding to human EMP2 and/or murine EMP2 are provided. Competition for binding to EMP2 or a portion of EMP2 by two or more anti-EMP2 antibodies may be determined by any suitable technique, as is known in the art.

Competition in the context of the present invention refers to any detectably significant reduction in the propensity of an antibody of the invention (e.g. KS49, KS41, KS83, or KS89) to bind its particular binding partner, e.g. EMP2, in the presence of the test compound. Typically, competition means an at least about 10-100% reduction in the binding of an antibody of the invention to EMP2 in the presence of the competitor, as measured by standard techniques such as ELISA or Biacore® assays. In certain embodiments, there is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 98% inhibition. In certain embodiments, there is 100% inhibition. Thus, for example, it is possible to set criteria for competitiveness wherein at least about 10% relative inhibition is detected; at least about 15% relative inhibition is detected; or at least about 20% relative inhibition is detected before an antibody is considered sufficiently competitive. In cases where epitopes belonging to competing antibodies are closely located in an antigen, competition may be marked by greater than about 40% relative inhibition of EMP2 binding (e.g., at least about 45% inhibition, such as at least about 50% inhibition, for instance at least about 55% inhibition, such as at least about 60% inhibition, for instance at least about 65% inhibition, such as at least about 70% inhibition, for instance at least about 75% inhibition, such as at least about 80% inhibition, for instance at least about 85% inhibition, such as at least about 90% inhibition, for instance at least about 95% inhibition, or higher level of relative inhibition).

In some cases, one or more of the components of the competitive binding assays are labeled.

It may also be the case that competition may exist between anti-EMP2 antibodies with respect to more than one of EMP2 epitope, and/or a portion of EMP2, e.g. in a context where the antibody-binding properties of a particular region of EMP2 are retained in fragments thereof, such as in the case of a well-presented linear epitope located in various tested fragments or a conformational epitope that is presented in sufficiently large EMP2 fragments as well as in EMP2.

Assessing competition typically involves an evaluation of relative inhibitory binding using an antibody of the invention, EMP2 (either human or murine or both), and the test molecule. Test molecules can include any molecule, including other antibodies, small molecules, peptides, etc. The compounds are mixed in amounts that are sufficient to make a comparison that imparts information about the selectivity and/or specificity of the molecules at issue with respect to the other present molecules.

The amounts of test compound, EMP2 and antibodies of the invention may be varied. For instance, for ELISA assessments about 5-50 μg (e.g., about 10-50 μg, about 20-50 μg, about 5-20 μg, about 10-20 μg, etc.) of the anti-EMP2 antibody and/or EMP2 targets are required to assess whether competition exists. Conditions also should be suitable for binding. Typically, physiological or near-physiological conditions (e.g., temperatures of about 20-40° C., pH of about 7-8, etc.) are suitable for anti-EMP2:EMP2 binding.

Often competition is marked by a significantly greater relative inhibition than about 5% as determined by ELISA and/or FACS analysis. It may be desirable to set a higher threshold of relative inhibition as a criteria/determinant of what is a suitable level of competition in a particular context (e.g., where the competition analysis is used to select or screen for new antibodies designed with the intended function of blocking the binding of another peptide or molecule binding to EMP2 (e.g., the natural binding partners of EMP2 or naturally occurring anti-EMP2 antibody).

In some embodiments, the anti-EMP2 antibody of the present invention specifically binds to one or more residues or regions in EMP2 but also does not cross-react with other proteins with homology to EMP2.

Typically, a lack of cross-reactivity means less than about 5% relative competitive inhibition between the molecules when assessed by ELISA and/or FACS analysis using sufficient amounts of the molecules under suitable assay conditions.

The disclosed antibodies may find use in blocking a ligand-receptor interaction or inhibiting receptor component interaction. The anti-EMP2 antibodies of the invention may be “blocking” or “neutralizing.” A “neutralizing antibody” is intended to refer to an antibody whose binding to EMP2 results in inhibition of the biological activity of EMP2, for example its capacity to interact with ligands, enzymatic activity, and/or signaling capacity Inhibition of the biological activity of EMP2 can be assessed by one or more of several standard in vitro or in vivo assays known in the art.

Inhibits binding” or “blocks binding” (for instance when referring to inhibition/blocking of binding of a EMP2 binding partner to EMP2) encompass both partial and complete inhibition/blocking. The inhibition/blocking of binding of a EMP2 binding partner to EMP2 may reduce or alter the normal level or type of cell signaling that occurs when a EMP2 binding partner binds to EMP2 without inhibition or blocking Inhibition and blocking are also intended to include any measurable decrease in the binding affinity of a EMP2 binding partner to EMP2 when in contact with an anti-EMP2 antibody, as compared to the ligand not in contact with an anti-EMP2 antibody, for instance a blocking of binding of a EMP2 binding partner to EMP2 by at least about 10%, 20%, 30%, 40%, 50%, 60%, 70%, 80%, 90%, 99%, or 100%.

The present invention further provides methods for producing the disclosed anti-EMP2 antibodies. These methods encompass culturing a host cell containing isolated nucleic acid(s) encoding the antibodies of the invention. As will be appreciated by those in the art, this can be done in a variety of ways, depending on the nature of the antibody. In some embodiments, in the case where the antibodies of the invention are full length traditional antibodies, for example, a heavy chain variable region and a light chain variable region under conditions such that an antibody is produced and can be isolated.

In general, nucleic acids are provided that encode the antibodies of the invention. Such polynucleotides encode for both the variable and constant regions of each of the heavy and light chains, although other combinations are also contemplated by the present invention in accordance with the compositions described herein. The present invention also contemplates oligonucleotide fragments derived from the disclosed polynucleotides and nucleic acid sequences complementary to these polynucleotides.

The polynucleotides can be in the form of RNA or DNA. Polynucleotides in the form of DNA, cDNA, genomic DNA, nucleic acid analogs, and synthetic DNA are within the scope of the present invention. The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence, which sequence, as a result of the redundancy or degeneracy of the genetic code, encodes the same polypeptides as the DNA provided herein.

In some embodiments, nucleic acid(s) encoding the antibodies of the invention are incorporated into expression vectors, which can be extrachromosomal or designed to integrate into the genome of the host cell into which it is introduced. Expression vectors can contain any number of appropriate regulatory sequences (including, but not limited to, transcriptional and translational control sequences, promoters, ribosomal binding sites, enhancers, origins of replication, etc.) or other components (selection genes, etc.), all of which are operably linked as is well known in the art. In some cases two nucleic acids are used and each put into a different expression vector (e.g. heavy chain in a first expression vector, light chain in a second expression vector), or alternatively they can be put in the same expression vector. It will be appreciated by those skilled in the art that the design of the expression vector(s), including the selection of regulatory sequences may depend on such factors as the choice of the host cell, the level of expression of protein desired, etc.

In general, the nucleic acids and/or expression can be introduced into a suitable host cell to create a recombinant host cell using any method appropriate to the host cell selected (e.g., transformation, transfection, electroporation, infection), such that the nucleic acid molecule(s) are operably linked to one or more expression control elements (e.g., in a vector, in a construct created by processes in the cell, integrated into the host cell genome). The resulting recombinant host cell can be maintained under conditions suitable for expression (e.g. in the presence of an inducer, in a suitable non-human animal, in suitable culture media supplemented with appropriate salts, growth factors, antibiotics, nutritional supplements, etc.), whereby the encoded polypeptide(s) are produced. In some cases, the heavy chains are produced in one cell and the light chain in another.

Mammalian cell lines available as hosts for expression are known in the art and include many immortalized cell lines available from the American Type Culture Collection (ATCC), Manassas, Va. including but not limited to Chinese hamster ovary (CHO) cells, HEK 293 cells, NSO cells, HeLa cells, baby hamster kidney (BHK) cells, monkey kidney cells (COS), human hepatocellular carcinoma cells (e.g., Hep G2), and a number of other cell lines. Non-mammalian cells including but not limited to bacterial, yeast, insect, and plants can also be used to express recombinant antibodies. In some embodiments, the antibodies can be produced in transgenic animals such as cows or chickens.

### Methods of Treatment

Antibody Compositions for In Vivo Administration

Formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. For example, it may be desirable to provide antibodies with other specificities. Alternatively, or in addition, the composition may comprise a cytotoxic agent, cytokine, growth inhibitory agent and/or small molecule antagonist. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration should be sterile, or nearly so. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Administrative Modalities

The antibodies and chemotherapeutic agents of the invention are administered to a subject, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Intravenous or subcutaneous administration of the antibody is preferred.

In certain aspects, the antibodies and chemotherapeutic agents of this invention are administered to a subject with cancer. In certain aspects, the antibodies and chemotherapeutic agents of the invention are administered to a subject with endometrial cancer.

In certain aspects, the antibodies and chemotherapeutic agents of this invention are administered to a subject with an angiogenic disorder. In certain aspects, the antibodies and chemotherapeutic agents of this invention are administered to a subject with a disease characterized by angiogenesis. In certain aspects, the antibodies and chemotherapeutic agents of this invention are administered to a subject with a disease characterized by neovascularization.

Treatment Modalities

In the methods of the invention, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling.

In addition to these positive therapeutic responses, the subject undergoing therapy may experience the beneficial effect of an improvement in the symptoms associated with the disease.

Such a response may persist for at least 4 to 8 weeks, or sometimes 6 to 8 weeks, following treatment according to the methods of the invention. Alternatively, an improvement in the disease may be categorized as being a partial response. By “partial response” is intended at least about a 50% decrease in all measurable tumor burden (i.e., the number of malignant cells present in the subject, or the measured bulk of tumor masses or the quantity of abnormal monoclonal protein) in the absence of new lesions, which may persist for 4 to 8 weeks, or 6 to 8 weeks.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the anti-EMP2 antibodies used in the present invention depend on the disease or condition to be treated and may be determined by the persons skilled in the art.

An exemplary, non-limiting range for a therapeutically effective amount of an anti-EMP2 antibody used in the present invention is about 0.1-100 mg/kg, such as about 0.1-50 mg/kg, for example about 0.1-20 mg/kg, such as about 0.1-10 mg/kg, for instance about 0.5, about such as 0.3, about 1, or about 3 mg/kg. In another embodiment, he antibody is administered in a dose of 1 mg/kg or more, such as a dose of from 1 to 20 mg/kg, e.g. a dose of from 5 to 20 mg/kg, e.g. a dose of 8 mg/kg.

A medical professional having ordinary skill in the art may readily determine and prescribe the effective amount of the pharmaceutical composition required. For example, a physician or a veterinarian could start doses of the medicament employed in the pharmaceutical composition at levels lower than that required in order to achieve the desired therapeutic effect and gradually increase the dosage until the desired effect is achieved.

In certain embodiments, the anti-EMP2 antibody is co-administered with a VEGF inhibitor. In certain embodiments, the VEGF inhibitor is bevacizumab, pazopanib, sorafenib, sunitinib, vandeteanib, cabozantinib, ponatinib, axitinib, aflibercept, or a combination or deriative thereof.

The term “co-administration” refers to the administration of an anti-EMP2 antibody and a VEGF inhibitor as one single formulation or as two separate formulations (one for the anti-EMP2 antibody and one for the VEGF inhibitor). The co-administration can be simultaneous or sequential in either order, wherein there is a time period while both (or all) active agents simultaneously exert their biological activities. As such, the “co-administration” of an anti-EMP2 and antibody and a VEGF inhibitor refers to a combination therapy of two or more chemical or biological substances involving an anti-EMP2 and antibody and a VEGF inhibitor. In sequential administration of an anti-EMP2 and antibody and a VEGF inhibitor, both agents can exert their biological activities at the same time. Alternatively, in sequential administration of an anti-EMP2 and antibody and a VEGF inhibitor, the agents can exert their biological activities at the same time.

In certain embodiments, the time between sequential administrations of an anti-EMP2 and antibody and a VEGF inhibitor can be less than 10 minutes, approximately 30 minutes, between 30 minutes and 1 hour, between 1 hour and 2 hours, between 2 hours and 5 hours, between 5 hours and 10 hours, between 6 hours and 12 hours, between 12 hours and 24 hours, approximately 1 day, approximately 2 days, approximately 3 days, approximately 4 days, approximately 5 days, approximately 6 days, approximately 7 days, approximately 8 days, approximately 9 days, approximately 10 days, approximately 11 days, approximately 12 days, approximately 13 days, approximately 14 days, approximately 15 days, approximately 16 days, approximately 17 days, approximately 18 days, approximately 19 days, approximately 20 days, approximately 21 days, approximately 22 days, approximately 23 days, approximately 24 days, approximately 25 days, approximately 26 days, approximately 27 days, approximately 28 days, approximately 29 days, approximately 30 days, approximately 35 days, approximately 40 days, or approximately 45 days.

In certain embodiments, the anti-EMP2 antibody and a VEGF inhibitor are co-administered approximately once a day, once every 7 days, once every 14 days, once every 28 days, once every 30 days, once every 45 days, once every 60 days, once every 90 days.

In one embodiment, the anti-EMP2 antibody is administered by infusion in a weekly dosage of from 10 to 500 mg/kg such as from 200 to 400 mg/kg. Such administration may be repeated, e.g., 1 to 8 times, such as 3 to 5 times. The administration may be performed by continuous infusion over a period of from 2 to 24 hours, such as from 2 to 12 hours.

In one embodiment, the anti-EMP2 antibody is administered by slow continuous infusion over a long period, such as more than 24 hours, if required to reduce side effects including toxicity.

In one embodiment the anti-EMP2 antibody is administered in a weekly dosage of from 250 mg to 2000 mg, such as for example 300 mg, 500 mg, 700 mg, 1000 mg, 1500 mg or 2000 mg, for up to 8 times, such as from 4 to 6 times. The administration may be performed by continuous infusion over a period of from 2 to 24 hours, such as from 2 to 12 hours. Such regimen may be repeated one or more times as necessary, for example, after 6 months or 12 months. The dosage may be determined or adjusted by measuring the amount of compound of the present invention in the blood upon administration by for instance taking out a biological sample and using anti-idiotypic antibodies which target the antigen binding region of the anti-EMP2 antibody.

In a further embodiment, the anti-EMP2 antibody is administered once weekly for 2 to 12 weeks, such as for 3 to 10 weeks, such as for 4 to 8 weeks.

In one embodiment, the anti-EMP2 antibody is administered by maintenance therapy, such as, e.g., once a week for a period of 6 months or more.

In one embodiment, the anti-EMP2 antibody is administered by a regimen including one infusion of an anti-EMP2 antibody followed by an infusion of an anti-EMP2 antibody conjugated to a radioisotope. The regimen may be repeated, e.g., 7 to 9 days later.

As non-limiting examples, treatment according to the present invention may be provided as a daily dosage of an antibody in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

In some embodiments the anti-EMP2 antibody is used in combination with one or more additional therapeutic agents, e.g. a chemotherapeutic agent or an anti-angiogenic agent. Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase; antibodies which bind to proteins overexpressed in cancers and other inhibitors of proteins or enzymes known to be upregulated, over-expressed or activated in cancers, the inhibition of which downregulates cell replication.

In some embodiments, the antibodies of the invention can be used prior to, concurrent with, or after treatment with any of the chemotherapeutic agents described herein or known to the skilled artisan at this time or subsequently.

In certain embodiments the anti-angiogenic agent is, for example, bevacizumab, itraconazole, carboxyamidotriazole, TNP-470, CM101, IFN-α, IL-12, platelet factor-4, suramin, SU5416, thrombospondin, VEGFR antagonists, angiostatic steroids+heparin, Cartilage-Derived Angiogenesis Inhibitory Factor, matrix metalloproteinase inhibitors, angiostatin, endostatin, 2-methoxyestradiol, tecogalan, tetrathiomolybdate, thalidomide, thrombospondin, prolactin, VEGF αVβ3 inhibitors, linomide, and tasquinimod.

### Efficacy of Methods Described Herein

In certain aspects of this invention, efficacy of anti-EMP2 therapy is measured by decreased serum concentrations of tumor specific markers, increased overall survival time, decreased tumor size, cancer remission, decreased metastasis marker response, and decreased chemotherapy adverse affects.

In certain aspects of this invention, efficacy is measured with companion diagnostic methods and products. Companion diagnostic measurements can be made before, during, or after anti-EMP2 treatment.

In some embodiments, the antibodies of the invention can be used to treat or diagnose a non-neoplastic condition. As used herein, a “non-neoplastic condition” refers to diseases not being or not caused by neoplasms.

### Companion Diagnostics

In other embodiments, this disclosure relates to companion diagnostic methods and products. In one embodiment, the companion diagnostic method and products can be used to monitor the treatment of uterine cancer, specifically endometrial cancer, as described herein. In some embodiments, the companion diagnostic methods and products include molecular assays to measure levels of proteins, genes or specific genetic mutations. Such measurements can be used, for example, to predict whether anti-EMP2 therapy will benefit a specific individual, to predict the effective dosage of anti-EMP2 therapy, to monitor anti-EMP2 therapy, adjust anti-EMP2 therapy, tailor the anti-EMP2 therapy to an individual, and track cancer progression and remission. Such measurements can also be used, for example, to predict whether anti-EMP2 and VEGF inhibitor co-administration therapy will benefit a specific individual, to predict the effective dosage of an anti-EMP2 and VEGF inhibitor co-administration therapy, to monitor an anti-EMP2 and VEGF inhibitor co-administration therapy, adjust an anti-EMP2 and VEGF inhibitor co-administration therapy, tailor the anti-EMP2 and VEGF inhibitor co-administration therapy to an individual, and track cancer progression and remission.

In some embodiments, the companion diagnostic can be used to monitor a combination therapy.

In some embodiments, the companion diagnostic can include an anti-EMP2 antibody described herein.

In some embodiments, the companion diagnostic can be used before, during, or after anti-EMP2 therapy.

### Articles of Manufacture

In other embodiments, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the antibody. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

In certain embodiments, an anti-EMP2 antibody and VEGF inhibitor are combined in a single container. In certain embodiments, an anti-EMP2 antibody and VEGF inhibitor are in separate containers and are administered separately. In certain embodiments, the anti-EMP2 antibody and VEGF inhibitor are stored in separate containers and are combined before administration.

The following references are incorporated by reference in their entirety.
Travasso, R. D. M., Corvera Pointe., Castro, M., Rodrguez-Manzaneque, L. C. & Hernandez-Machado, A. Tumor Angiogenesis and Vascular Patterning: A Mathematical Model. PLoS One 6, e19989 (2011).
Park, J. E., Keller, H. A. & Ferrara, N. The vascular endothelial growth factor isoforms (VEGF): differential deposition into the subepithelial extracellular matrix and bioactivity of extracellular matrixbound VEGF. Mol. Biol. Cell 4, 1317-1326 (1993).
Ferrara, N. VEGF and the quest for tumour angiogenesis factors. Nat Rev Cancer 2, 795-803 (2002).
Ferrara, N., Hillan, K. J., Gerber, H.-P. & Novotny, W. Discovery and development of bevacizumab, an anti-VEGF antibody for treating cancer. Nat Rev Drug Discov 3, 391-400 (2004).
Cebe-Suarez, S., Zehnder-Fjallman, A. & Ballmer-Hofer, K. The role of VEGF receptors in angiogenesis; complex partnerships. Cellular and Molecular Life Sciences 63, 601-615, doi: 1 0.1 007/s000 18-005-5426-3 (2006).
Tang, N. et al. Loss of HIF-1[alpha] in endothelial cells disrupts a hypoxia-driven VEGF autocrine loop necessary for tumorigenesis. Cancer Cell 6, 485-495 (2004).
Inoue, M., Hager, 1. H., Ferrara, N., Gerber, H.-P. & Hanahan, D. VEGF-A has a critical, nonredundant role in angiogenic switching and pancreatic 2 cell carcinogenesis. Cancer Cell 1, 193-202 (2002).
Robinson, C. J. & Stringer, S. E. The splice variants of vascular endothelial growth factor (VEGF) and their receptors. J Cell Sci 114, 853-865 (2001). 26 Bates, D. O. et al. The endogenous anti-angiogenic family of splice variants of VEGF, VEGFxxxb, are down-regulated in pre-eclamptic placentae at term. Clin Sci (London) 110, 575-585 (2006).
Zerefos, P. G. et al. Characterization of the human urine proteome by preparative electrophoresis in combination with 2-DE. Proteomics 6, 4346-4355, doi:IO.1002/pmic.200500671 [doi] (2006).
Wadehra, M. et al. Knockdown of the tetraspan protein epithelial membrane protein-2 inhibits implantation in the mouse. Dev. Biol. 292, 430-441 (2006).
Wadehra, M. et al. Epithelial membrane protein-2 regulates surface expression of alphavbeta3 integrin in the endometrium. Dev. Biol. 287, 336-345 (2005).
Wadehra, M. et al. Expression of epithelial membrane protein-2 is associated with endometrial adenocarcinoma of unfavorable outcome. Cancer 107, 90-98, doi:10.1002/cncr.21957 [doi] (2006).

Arnaoutova, 1. & Kleinman, H. K. In vitro angiogenesis: endothelial cell tube formation on gelled basement membrane extract. Nat. Protocols 5, 628-635 (2010).
Merdzhanova, G. et al. The transcription factor E2F 1 and the SR protein SC35 control the ratio of proangiogenic versus antiangiogenic isoforms of vascular endothelial growth factor-A to inhibit neovascularization in vivo. Oncogene 29, 5392-5403, doi:hypertext transfer protocol://www.nature.com/onc/journal/v29/n39/suppinfo/onc20 1 0281 s 1.html (2010).
Wadehra, M., Braun, 1. & Goodglick, L. One Step RT-PCR for Screening Microdissected Tissue. Biotechniques 32, 242-247 (2002).
Morales, S. et al. Collagen Gel Contraction by ARPE-19 is mediated by a FAK-Src dependent pathway. Experimental Eye Research 85, 790-798 (2007).
Barnett, S. F. et al. Identification and characterization of pleckstrin-homology-domain-dependent and isoenzyme-specific Akt inhibitors. Biochem J 385, 399-408, doi: 10.1042/BJ20041140 [doi] BJ20041140 [pii] (2005).
Wang, M. Y. et al. Mammalian target of rap amy cin inhibition promotes response to epidermal growth factor receptor kinase inhibitors in PTEN-deficient and PTEN-intact glioblastoma cells. Cancer Res 66, 7864-7869, doi:66/1617864 [pii] 10.1158/0008-5472.CAN-04-4392 [doi] (2006).
Lu, K. V. et al. Fyn and SRC are effectors of oncogenic epidermal growth factor receptor signaling in glioblastoma patients. Cancer Res 69, 6889-6898, doi:0008-5472.CAN-09-0347 [pii] 10.1158/0008-5472. CAN-09-0347 [doi] (2009).
Shimazaki, K. et al. Diabodies targeting epithelial membrane protein 2 reduce tumorigenicity of human endometrial cancer cell lines. Clin Cancer Res 14, 7367-7377, doi: 14/2217367 [pii]10.1158/1078-0432.CCR-08-1016 [doi] (2008).
Mancuso, M. R. et al. Rapid vascular regrowth in tumors after reversal of VEGF inhibition. The Journal of Clinical Investigation 116, 2610-2621 (2006).
Kadenhe-Chiweshe, A. et al. Sustained VEGF Blockade Results in Microenvironmental Sequestration of VEGF by Tumors and Persistent VEGF Receptor-2 Activation. Molecular Cancer Research 6, 1-9, doi: 1 0.1158/1541-7786.mcr-07-0101 (2008).
Kilarski, W. W., Samolov, B., Peters son, L., Kvanta, A. & Gerwins, P. Biomechanical regulation of blood vessel growth during tissue vascularization. Nat Med 15, 657-664, doi:hypertext transfer protocol://www.nature.com/nm/joumal/v 1 5/n6/suppinfo/nm. 1985 S 1.html (2009).
Fu, M. et al. Epithelial Membrane Protein-2 Promotes Endometrial Tumor Formation through Activation of FAK and Src. PLoS One 6, e 19945 (2011).
Habeeb, O. et al. Epithelial membrane protein-2 expression is an early predictor of endometrial cancer development. Cancer 116, 4718-4726, doi:10.1002/cncr.25259 [doi] (2010).
Fu, M. et al. Epithelial membrane protein-2 is a novel therapeutic target in ovarian cancer. Clin Cancer Res 16, 3954-3963, doi: 16/15/3954 [pii] 10.1158/1078-0432.CCR-10-0368 [doi] (2010).
Dvorak, H. F. Vascular Permeability Factor/Vascular Endothelial Growth Factor: A Critical Cytokine in Tumor Angiogenesis and a Potential Target for Diagnosis and Therapy. Journal of Clinical Oncology 20, 4368-4380, doi: 10. 1200/jco.2002.10.088 (2002).
Mahimainathan, L., Ghosh-Choudhury, N., Venkatesan, B. A., Danda, R. S. & Choudhury, G. G. EGF stimulates mesangial cell mitogenesis via PI3-kinase-mediated MAPK-dependent and AKT kinase independent manner: involvement of c-fos and p27Kip 1. American Journal of Physiology—Renal Physiology 289, F72-F82, doi:10.1152/ajprenal.00277.2004 (2005).
Vivanco, I. & Sawyers, C. L. The phosphatidylinositol3-Kinase AKT pathway in human cancer. Nat Rev Cancer 2, 489-501, doi:10.1038/nrc839 [doi] nrc839 [pii] (2002).
Wadehra, M, Iyer, R., Goodglick, L. & Braun, J. The tetraspan protein epithelial membrane protein-2 interacts with beta1 integrins and regulates adhesion. J Bioi Chern 277, 41094-41100 (2002).
Aplin, A. E., Short, S. M. & Juliano, R. L. Anchorage-dependent regulation of the mitogen-activated protein kinase cascade by growth factors is supported by a variety of integrin alpha chains. J. Biol. Chern. 274, 31223-31228 (1999).
Leite de Oliveira, R., Hamm, A. & Mazzone, M. Growing tumor vessels: More than one way to skin a cat-Implications for angiogenesis targeted cancer therapies. Molecular Aspects of Medicine 32, 71-87, doi: 10.1 016/j.mam.2011.04.001 (2011).
Zagzag, D. et al. Hypoxia-inducible factor 1 and VEGF upregulate CXCR4 in glioblastoma: implications for angiogenesis and glioma cell invasion. Lab Invest 86, 1221-1232 (2006).

The invention is now described with reference to the following examples. These examples are provided for the purpose of illustration only and the invention should in no way be construed as being limited to these examples but rather should be construed to encompass any and all variations which become evident as a result of the teachings provided herein.

Whereas, particular embodiments of the invention have been described herein for purposes of description, it will be appreciated by those skilled in the art that numerous variations of the details may be made without departing from the invention as described in the appended claims.

### EXAMPLES

### Example 1

### EMP2 IgG1 Improves Endometrial Cancer Survival

To determine the efficacy of EMP2 IgG1 for endometrial cancer, subcutaneous xenografts using the HEC1a/EMP2 cell line were created.

The human endometrial adenocarcinoma cell line HEC-1A (HTB112, ATCC, Manassas, Va.) was cultured in McCoys media supplemented with 10% fetal calf serum at 37° C. in a humidified 5% CO2 incubator. Cell lines were used within 2 months after resuscitation of frozen aliquots and were authenticated based on viability, recovery, growth, morphology, and isoenzymology by the supplier. Stably transfected HEC-1A cells containing a human EMP2-GFP fusion protein or control GFP.

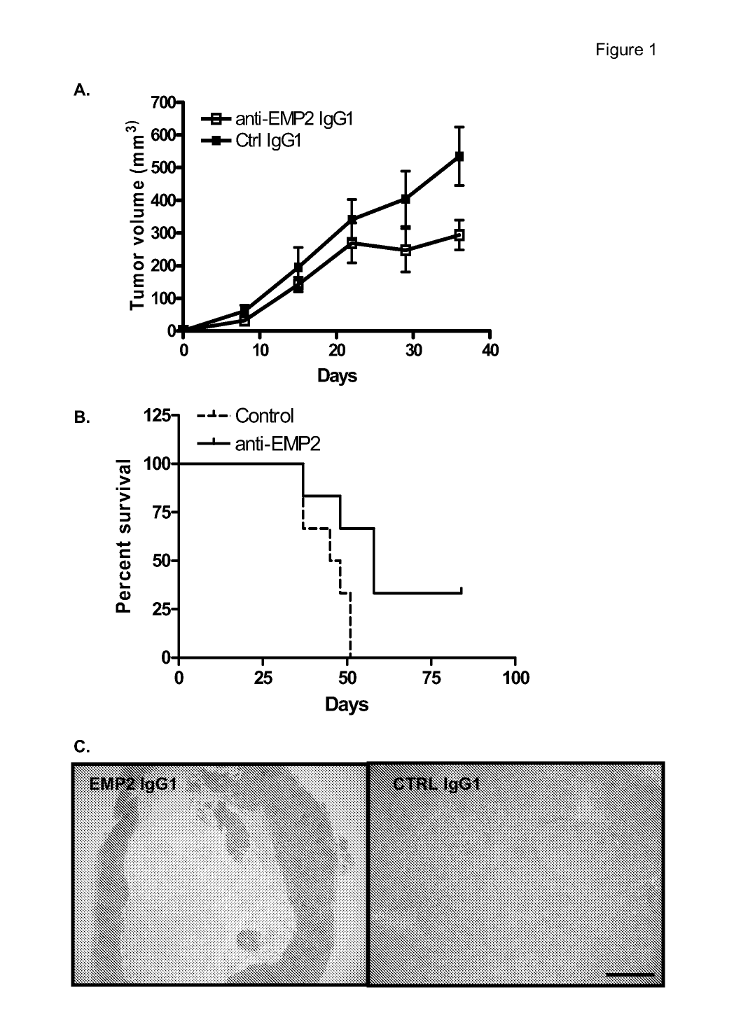

Systemic weekly injections of EMP2 IgG1 reduced tumor load compared to control IgG (FIG. 1A). This reduction in tumor load translated into a significant increase in survival for mice treated with EMP2 IgG 1 (FIG. 1B; p=0.02). Even after 84 days, surviving mice showed no measurable change in tumor size. Upon histologic examination, central necrosis was prominent after anti-EMP2 antibody treatment compared with the control (FIG. 1 C).

### Example 2

### Tumor Associated Vasculature

Tumors were created from endometrial cancer cells that overexpressed EMP2 (HEC1a/EMP2), expressed a vector control (HEC1a/V), or expressed a ribozyme to reduce its levels (HEC1a/RIBO).

Human umbilical vein endothelial cells (HUVEC) from passage 2 were purchased from BD Biosciences (San Diego, Calif.) and used between passages 3 and 7 for all experiments. Primary human coronary artery endothelial cells (HAECs) (gift from Dr. J. Berliner, UCLA) were also utilized. All endothelial cells were grown in complete MCDB-131 complete media (VEC Technologies, Renesselaer, N.Y.). Stably infected HEC-1A cells containing a non-targeting shRNA control or EMP2 specific shRNA (385; TRCN0000072385, 911; TRCN0000322911) in pLKO.1-puro were generated as per manufacturer's instructions (Sigma-Aldrich, St. Louis, Mo.).

Four to six-week-old nude BALB/c female mice were obtained from Charles River Laboratories (Wilmington, Mass.) and maintained at the University of California, Los Angeles. Animals were inoculated subcutaneously (s.c.) with 1×10⁶ HEC-IA/OE, HEC-IA1V, or HEC-IA/RlBO cells into the right and left shoulder flanks, respectively. Tumors were measured using calipers, and the volume calculated with the formula: length×width/2. Six mice were used per group. At day 30, tumors were excised, fixed in formalin, and then processed for hematoxylin and eosin staining by the Tissue Procurement Laboratory at UCLA or by Masson's trichrome stain (Dako, Carpinteria, Calif.).

In some experiments, 1×10⁶ HEC1a/EMP2 cells were suspended in 5% matrigel (BD Biosciences) and injected s.c. into the shoulder of female athymic mice. When tumors approached 4 mm², systemic treatments with 10 mg/kg dose of anti-EMP2 IgGI or control IgG (Sigma-Aldrich) were administered weekly via an intraperitoneal (i.p.) route. Tumor size was monitored, and mice euthanized once tumors approached 1.5 cm in diameter or became ulcerated. Tumors were isolated, fixed and processed for hematoxylin and eosin staining.

Samples were analyzed to quanitavely and identity tumor vasculature. Masson's trichrome staining was performed according the manufacturer's instructions (DAKO, Carpinteria, Calif.). Samples were deparaffinized and dehydrated in alcohol. In some experiments, after blocking in 1% normal goat serum, samples were incubated with 1:50 dilution of FITC-labeled L. esculentum lectin (1 mg/ml in 0.9% NaCl; Vector Laboratories).

Samples were then counterstained with DAPI and mounted. To quantitate CD34 expression, samples were deparaffinized and then incubated at 95° C. for 20 minutes in 0.1 M citrate, pH 6.0. Rat anti-CD34 (Abcam, Cambridge, Mass.) was used at a dilution of 1:25 as previously described followed by visualization using the Vector ABC kit (Vector Labs, Burlingame, Calif.) according to the manufacturer's instructions. The numbers of CD34-positive blood vessels in 6 high power fields (×200) were counted and averaged.

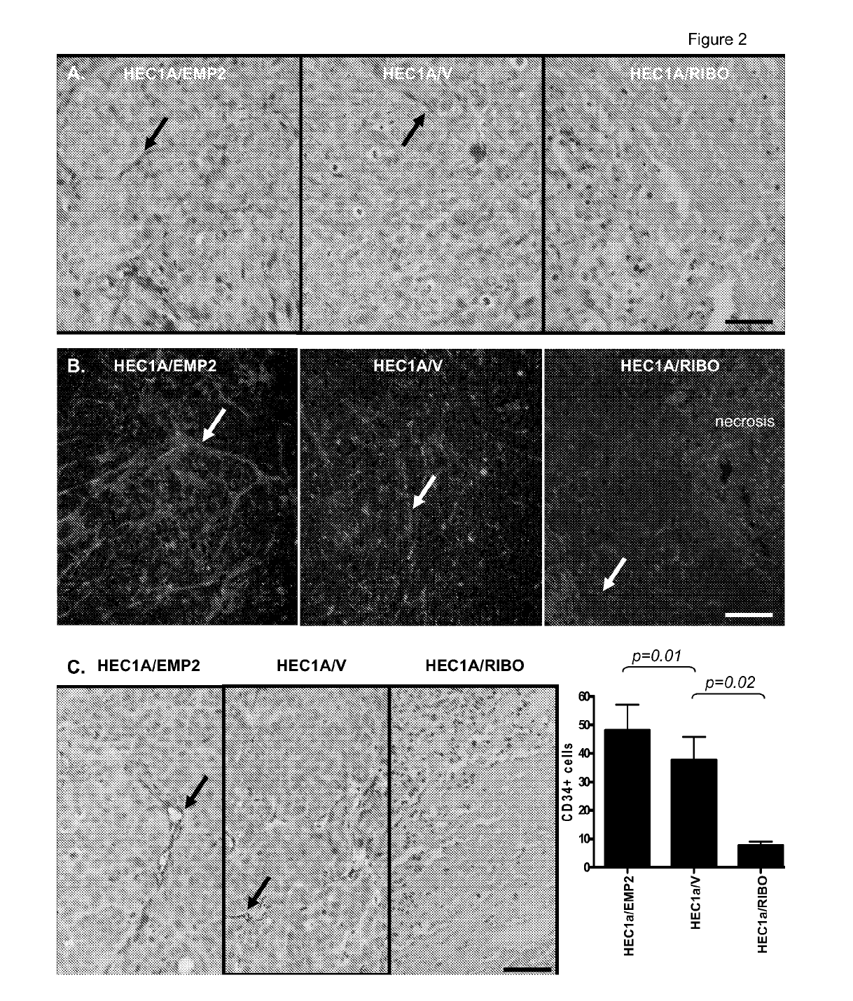

Masson's trichrome staining of xenografts suggested that EMP2 expression altered tumor vasculature (FIG. 2A). HEC1a1EMP2 tumors were highly vascularized, while tumors with reduced EMP2 (HEC1a/RIBO) levels formed small tumors with poor vasculature and large areas of necrosis.

Capillary like tube formation was performed. Briefly, coverslips were coated with reduced growth factor basement membrane (Geltrex; Invitrogen, Carlsbad, Calif.) and incubated at 37° C. for 30 min to promote jelling. 5×10⁵HUVECs were resuspended in cultured media from HECIa/EMP2, HEC1a/V, or HEC1a/RIBO cells. Conditioned media was typically prepared as follows: confluent cells were grown in 10% FBS-McCoys for 72 h or placed in a hypoxic chamber for up to 24 hours. Migration assays. HUVEC and HAEC migration assays were conducted in Boyden chambers as previously reported. Conditioned media was prepared as described above. In some experiments, cultured supernatants from tumor cells were treated with Bevacizumab (Genentech BioOncology, South San Francisco, Calif., USA). Bevacizumab was obtained from the pharmacy at University of California, Davis. Each condition was assayed in triplicate wells. “Scratch” wound closure assays were performed by creating a confluent monolayer of HUVEC cells. Using the tip of a Pasteur pipet, a scratch was created. Conditioned media from HEC 1 alEMP2, HEC1a/V, or HEC 1 a/RIB 0 cells were added to the wells. Three random measurements for each of three wounds were measured for each test condition. The experiment was repeated three times.

The cultured media was then collected and centrifuged to remove cell debris. In some experiments, HEC1a cells were treated with 100/lg/ml of the full-length EMP2 IgGI for 24 hours 13, or recombinant human VEGF (Sigma-Aldrich) was used as an inducer from 0-50 ng/mL. After 18 h, cells were stained with calcein AM and analyzed.

To confirm that EMP2 levels correlated with increased numbers of blood vessels, tumors were stained with Lycopersicon esculentum lectin, which binds uniformly to the luminal surface of the endothelium, and DAPI. HEC1a/EMP2 tumors showed increased tumor associated vasculature compared with the HEC1a/V tumors. Similar staining of HEC1a/RIBO tumors showed poor tumor vasculature with some background staining in areas of necrosis (FIG. 2B). Xenographs were also stained with CD34 antibodies with concordant results (FIG. 2C).

### Example 3

### EMP2 Expression Promotes Endothelial Cell Tube Formation

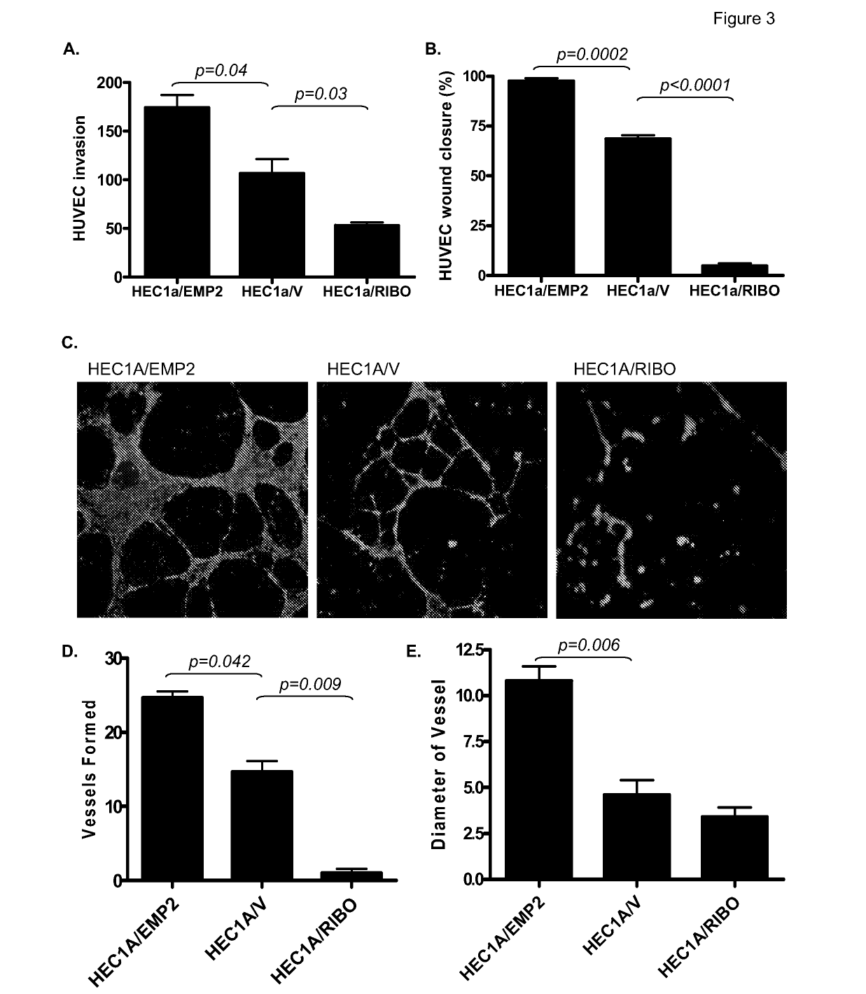

Several approaches were used to investigate whether and how EMP2 could regulate the behavior of endothelial cells. Initially, the chemotactic response of HUVECs to supernatants from EMP2 modified cell lines was tested using Boyden chambers. After 4-6 hrs, EMP2 tumor expression significantly enhanced directional migration compared to control cells (p=0.04; FIG. 3A). Reduction in EMP2 expression further reduced cell migration by two-fold over control cells (p=0.03). To confirm that EMP2 expression altered endothelial cell migration, a “scratch” test was performed on a confluent monolayer of HUVEC cells. Concordant with the previous results, an EMP2 dose-dependent response was also observed (FIG. 3B).

To determine whether EMP2 altered the functional behavior of endothelial cells, HUVEC cells were placed on a basement membrane matrix to induce capillary-like tube formation. Cells were incubated in cultured supernatants from HEC1a/EMP2, HEC1a/V, and HEC1a/RIBO (FIG. 3C). An EMP2-dependent response was observed in capillary like tube formation as HEC1a/EMP2 induced more tube formation and tubes with a greater diameter than HEC1a/V (FIG. 3D, E). Reduction in EMP2 expression in HEC1a cells further reduced the number of tubes formed compared with HEC1a/V, suggesting that EMP2 expression is necessary for endometrial tumor angiogenesis. This effect was also observed when EMP2 levels were reduced using shRNA (data not shown). No significant difference in HUVEC cell proliferation was observed within the experimental window (data not shown).

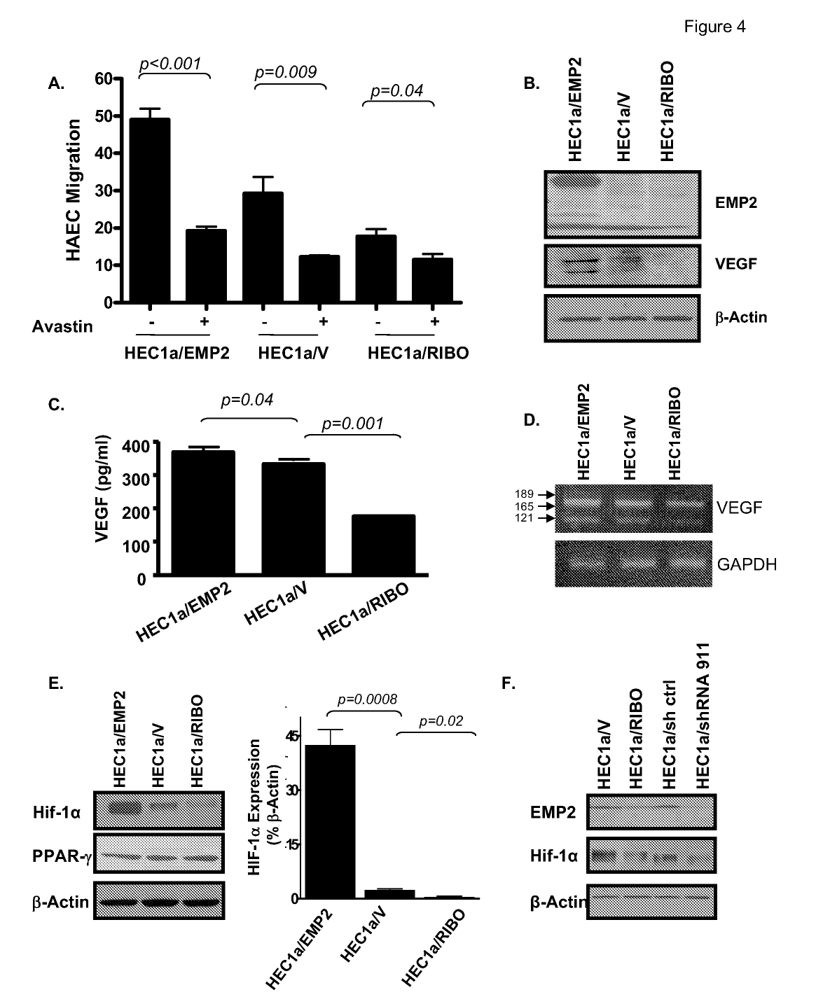

To determine if the effects of EMP2 on endothelial cells are cell type specific, similar experiments were performed on primary human aortic endothelial cells (HAEC). Using supernatant from HEC1a/EMP2, HEC1a/V, or HEC1a/RIBO as a chemoattractant, Boyden chamber assays were performed on HAEC. Similar to results using HUVEC cultures, tumor expression of EMP2 promoted HAEC invasion (FIG. 4A). These combined results suggest that EMP2 upregulation leads to an increase in proangiogenic events. Several cellular and molecular changes have been shown to promote tumor angiogenesis, with the most potent inducer being vascular endothelial growth factor (VEGF). In order to determine if VEGF contributed to HAEC invasion, tumor cell supernatants were incubated with Bevacizumab, a monoclonal antibody to VEGF. Treatment with Bevacizumab reduced HAEC migration to control levels, suggesting that EMP2 may regulate VEGF expression.

### Example 4

### EMP2 Regulates VEGF Expression

To determine if EMP2 expression altered VEGF expression and secretion, cells were placed in a hypoxic chamber for 24 hours. EMP2 expression directly correlated with VEGF protein levels (FIG. 4B) as well as with secreted protein (FIG. 4C). In contrast, reduction of EMP2 resulted in undetectable levels of VEGF by western and low levels of secreted protein. Western blot analysis was carried out by Cells were lysed in Laemmli buffer. Proteins were separated by SDS-PAGE, transferred to a nitrocellulose membrane (Amersham Biosciences), and stained with Ponceau S (Sigma-Aldrich, St. Louis, Mo.) to determine transfer efficiency. Membranes were blocked with 10% low fat milk in PBS containing 0.1% Tween 20 and probed with EMP2 antisera (1:1000), anti-VEGF (Santa Cruz Biotech), antiHIF-1a (1:800; BD Biosciences), anti-PPAR-y (Santa Cruz Biotech), anti-p-Src (Cell Signaling, Danvers). Protein bands were visualized using a horseradish peroxidase (HRP)-labeled secondary antibody (BD Biosciences) followed by chemiluminescence (ECL; Amersham Biosciences). Band intensities were quantified using the NIH program Image J as above. To account for loading variability β-actin was used to normalize each sample. At least three independent experiments were performed and, where indicated, the results were evaluated for statistical significance using a Student's t-test (unpaired, one-tail). A level of P<0.05 was considered to be statistically significant.

MA), anti-p-AKT (Cell Signaling), anti)76/S77p_FAK (1:500; BD Biosciences), or ˜-actin (Sigma-Aldrich).

To confirm these results, semi-quantitative RT-PCR was performed on HEC1a/EMP2, HEC1a/V, and HEC1a/RIBO cells. For RT-PCR analysis, total cellular RNAs were isolated using RNeasy mini kit (Qiagen, Valencia, Calif.). In all conditions, 1 μg of total RNA was reversed transcribed using oligo(dT) primers and Moloney murine leukaemia virus reverse transcriptase (Invitrogen). For VEGF amplification including all four splice variants, the PCR conditions and primers were utilized as previously described. Amplification of a GAPDH cDNA fragment was performed in a separate PCR reaction as described. PCR products were run on a 2% agarose gel and were visualized by ethidium bromide staining.

VEGF-A exists as multiple isoforms, which are generically referred to as VEGFxxx and result from the pre-mRNA alternative splicing of eight exons. Alternative splicing of VEGF-A was initially shown to generate four different isoforms with 121, 165, 189 and 206 amino acids (VEGF121, VEGF165, VEGF189, VEGF206, respective well). EMP2 levels directly increased the mRNA expression of several VEGF isoforms (VEGF165 and VEGF 121) while a reduction in EMP2 produced concordant results (FIG. 4D). Low levels of VEGF189 mRNA were observed in HEC1a/EMP2 cells, and no expression of VEGF206 was detected in any of the cell lines.

Several oncogenic as well as growth factor-driven pathways have been shown to regulate VEGF expression. As hypoxic conditions have been shown to trigger with many oncogenic signaling pathways, we initially investigated if the levels of EMP2 were sufficient to alter the hypoxic-induced transcription factor (HIF-1a) expression. Highest levels of HIF-1a express were directly correlated with the highest concentration of EMP2 expression under hypoxic conditions (FIG. 4E). HEC1a/EMP2 cells significantly induced HIF-1a expression compared to HEC1a/V cells (P=0.0008). Reciprocally, HEC1a/RIBO cells produced below detection levels of HIF-1a under the same conditions. To confirm that lower levels of EMP2 reduce HIF-1a expression, shRNA constructs were generated to reduce EMP2 expression (FIG. 4F).

In some experiments, inhibitors were added to determine the contribution of specific pathways to HIF-1a expression. HEC1a/EMP2 cells were treated with 10 μM of the kinase inhibitor Ly294002 (Cell Signaling), the EGFR inhibitor Erlotinib e; 10 μM, Genentech), or the Src family tyrosine kinase inhibitor Dasatinib e; 10 μM, Bristol-Myers Squibb). Efficacy of the inhibitors was tested at the manufacturer's recommended dosage, and potential toxicity was measured using trypan-blue exclusion. Samples were harvested and probed by SDS-PAGE/Western blot analysis as above FAK-Src small molecule inhibitor PP2 or the small molecule control PP3 16, 5f-1M of Akt inhibitor VIII (17; Calbiochem, San Diego, Calif.), 50 f-1M of the PI3

Similar to experiments with the ribozymes, EMP2 shRNA reduced HIF-1a levels compared to shRNA controls.

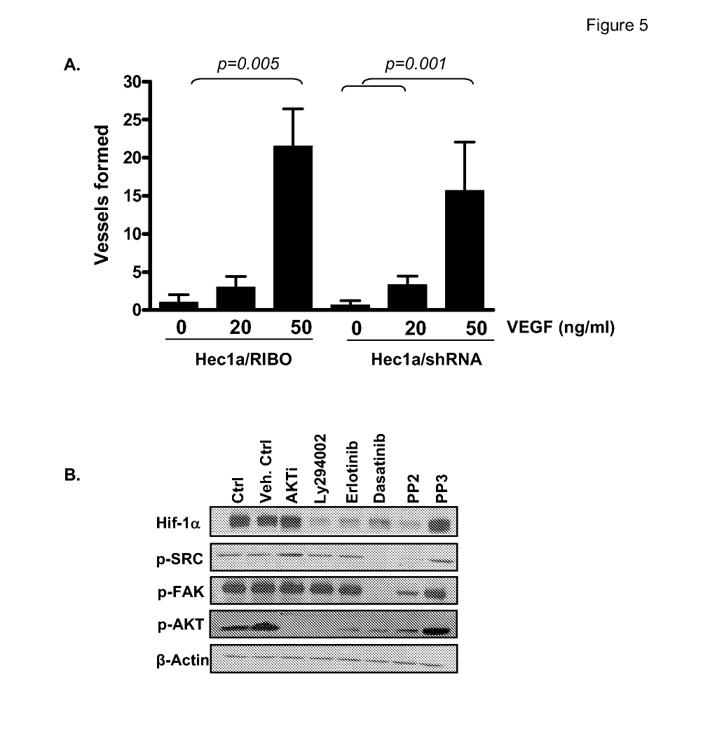

It was demonstrated that downregulation of EMP2 in the tumor cell reduced capillary formation and the motility of endothelial cells using coculture assays. This may be the result of reduced VEGF secretion by these cells or due to the expression of anti-angiogenic agents. To address this question, varying amounts of VEGF were added to supernatants from vehicle control or cells with reduced EMP2 levels (HEC1a/RIBO or HEC1a/shRNA 911). Exogenous VEGF was sufficient to induce HUVEC cell tube formation (FIG. 5A), suggesting that the absence of VEGF contributed to lack of tube formation.

### Example 5

### EMP2 Promotes HIF-1a Expression Through a Src Mediated Pathway

EMP2 has been shown to promote integrin mediated FAK and Src activation, therefore we next examined whether the EMP2-stimulated FAK I Src activation was directly correlated with increased HIF-1a expression. Using common inhibitors of AKT, PI3-kinase, EGFR, and Src tyrosine kinases, HEC1a/EMP2 cells were treated with these agents while in a hypoxic chamber. As shown in FIG. 5B, EMP2-induced overexpression of HIF-1a was reversed using Src inhibitors PP2 and dasatinib, respectively. Both erlotinib and Ly294002, a PI3-kinase inhibitor, also produced a similar effect, suggesting an overlap in signaling with EMP2. In contrast, AKTi did not alter HIF-1a expression in these cells (FIG. 5B).

### Example 6

### Anti-EMP2 Antibody Therapy Suppresses Neovascularization

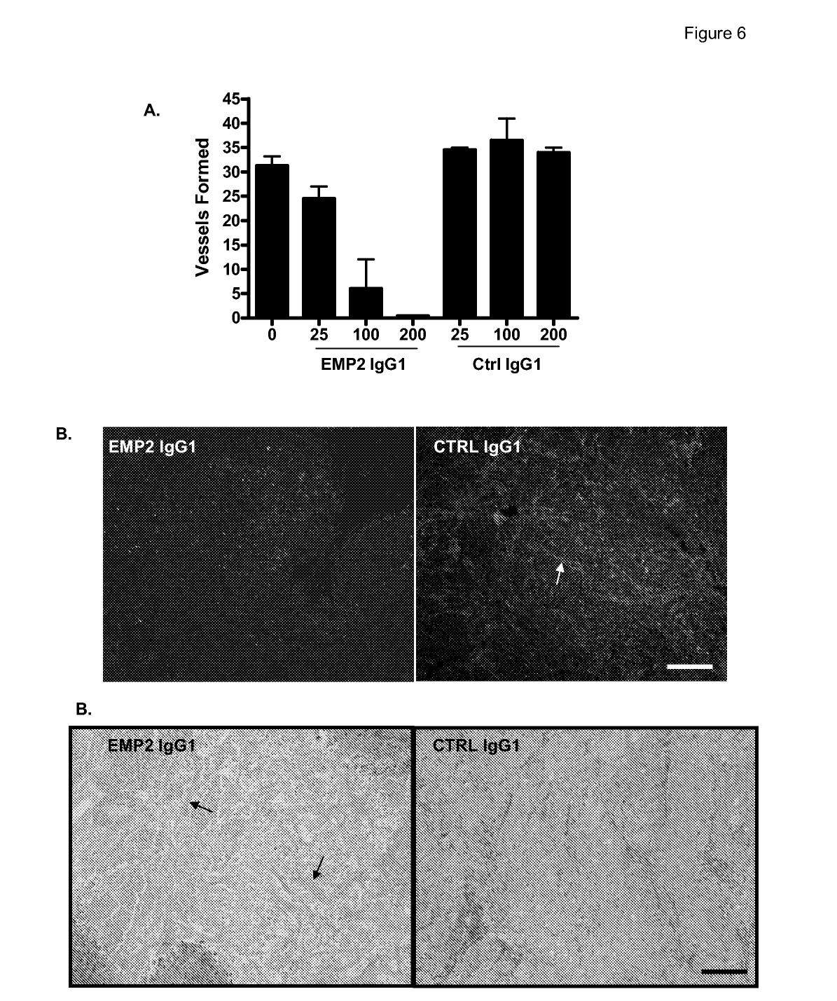

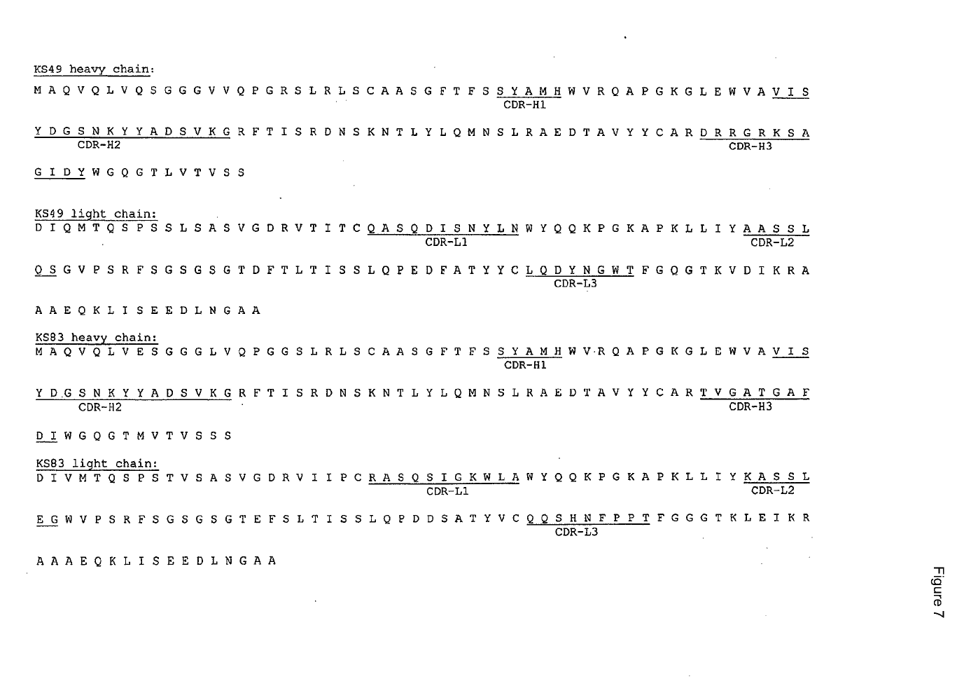

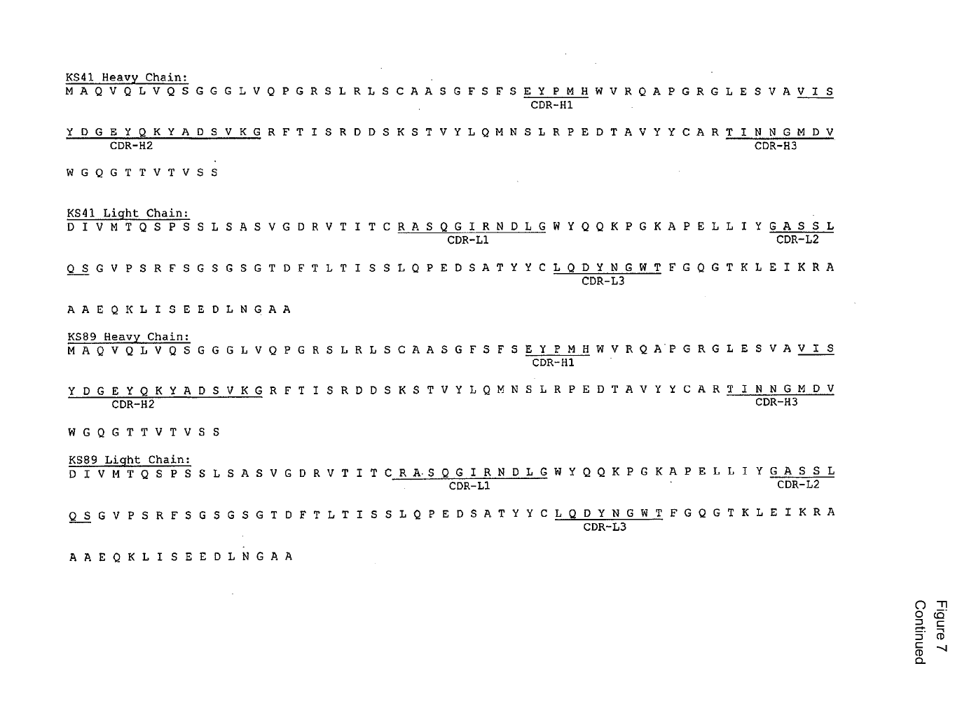

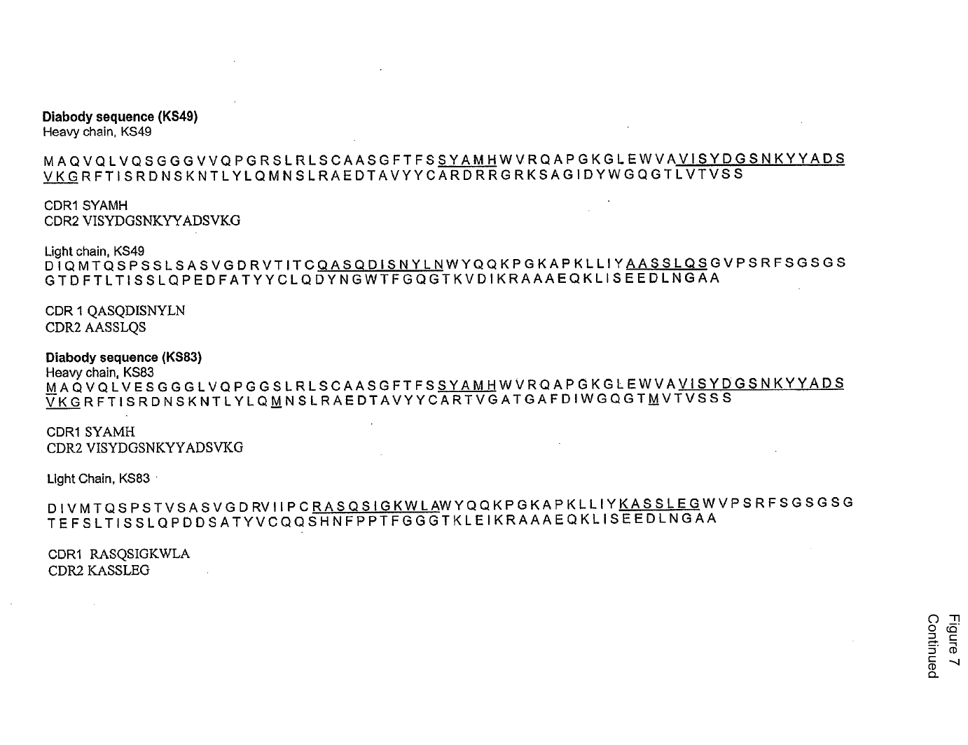

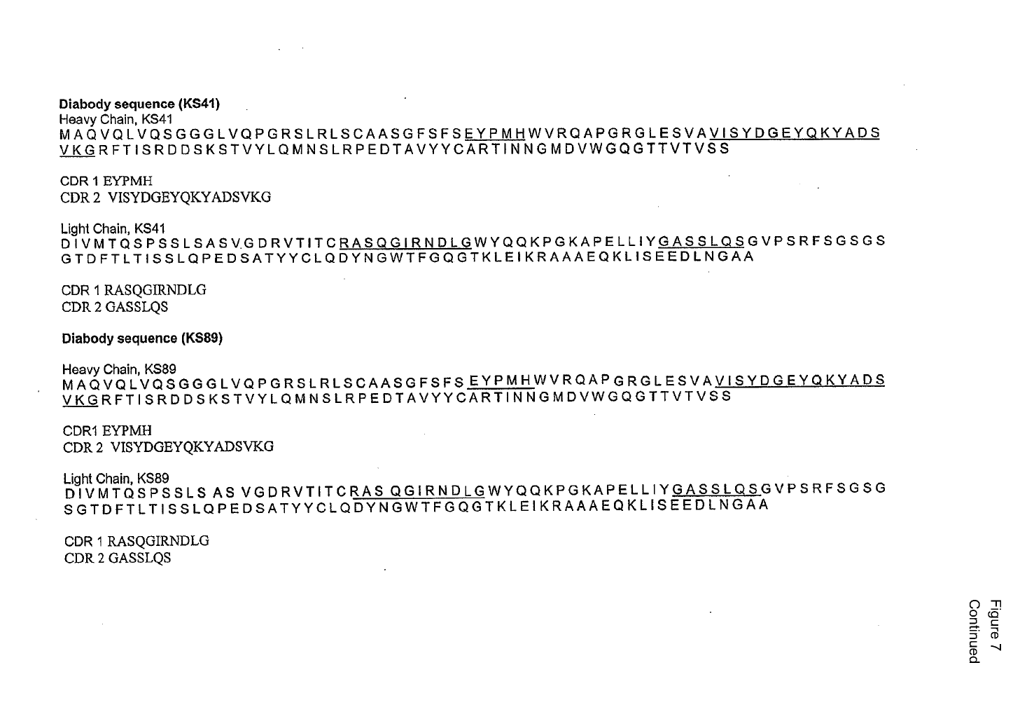

To further examine whether the reductions in tumor vascularity from EMP2 treatment were due to vessel regression or reduced angiogenesis, tumors were stained Masson's trichrome to detect the presence of collagen sleeves in necrotic areas (FIG. 6C). All treatment groups had scattered fragments of basement membrane (FIG. 6C, arrowheads), but empty basement membrane sleeves were more abundant in areas of necrosis after the anti-EMP2 antibody than control treatments. This suggests that blockade of EMP2 is a novel mechanism to reduce tumor neovascularization.

We claim:

1. A method of treating a patient for uterine cancer, the method comprising co-administering to the patient an effective amount of an anti-angiogenic agent or a chemotherapeutic agent and an effective amount of an anti-EMP2 antibody to a subject in need thereof, wherein
the anti-EMP2 antibody specifically binds to an epitope in the second extracellular loop of EMP2, and
the epitope in the second extracellular loop of EMP2 comprises the amino acid sequence DIHDKNAKFYPVTREGSYG.

2. The method of claim 1, wherein the anti-EMP2 antibody is a humanized or fully human antibody.

3. The method of claim 1, wherein the anti-EMP2 antibody is a diabody.

4. The method of claim 1, wherein the anti-EMP2 antibody is a triabody.

5. The method of claim 1, wherein the anti-EMP2 antibody is a minibody.

6. The method of claim 1, wherein the anti-EMP2 antibody is a single chain antibody.

7. The method of any of claims 1 to 6, wherein the effective amount of an anti-angiogenic agent or a chemotherapeutic agent and an effective amount of an anti-EMP2 antibody further comprises a physiological acceptable carrier or a pharmaceutically acceptable carrier.

8. The method of any of claims 1 to 7, wherein the anti-EMP2 antibody competes with an antibody comprising the heavy and light chain variable regions of a KS49, a KS41, a KS83, or a KS89 diabody.

9. The method of any of claims 1 to 8, wherein the antibody shares 90% amino acid identity with heavy and light chain variable regions of a KS49, a KS41, a KS83, or a KS89 diabody.

10. The method of any of claims 1 to 9, wherein the anti-angiogenic agent is a VEGF inhibitor.

11. The method of claim 10, wherein the VEGF inhibitor is an anti-VEGF antibody.

12. The method of claim 11, wherein the anti-VEGF antibody is bevacizumab.

13. The method of claim 11, wherein the anti-VEGF antibody is selected from a group comprising pazopanib, sorafenib, sunitinib, vandeteanib, cabozantinib, ponatinib, axitinib, and aflibercept.

14. The method of any of claims 1 to 9, wherein the chemotherapeutic agent is a DNA damaging chemotherapeutic agent.

15. The method of claim 14, wherein the DNA damaging chemotherapeutic agent is selected from a group comprising topoisomerase I inhibitors, topoisomerase II inhibitors, alkylating agents, DNA intercalators, free radical generators, and nucleoside mimetics.

16. The method of claim 15, wherein the topoisomerase I inhibitor is selected from a group comprising irinotecan, topotecan, camptothecin and analogs or metabolites thereof and doxorubicin.

17. The method of claim 15, wherein the topoisomerase II inhibitor is selected from a group comprising etoposide, teniposide, and daunorubicin.

18. The method of claim 15, wherein the alkylating agent is selected from a group comprising melphalan, chlorambucil, busulfan, thiotepa, ifosfamide, carmustine, lomustine, semustine, streptozocin, decarbazine, methotrexate, mitomycin C, and cyclophosphamide.

19. The method of claim 15, wherein the DNA intercalator is selected from a group comprising cisplatin, oxaliplatin, and carboplatin.

20. The method of claim 15, wherein the free radical generator is bleomycin.

21. The method of claim 15, wherein the nucleoside mimetic is selected from a group comprising 5-fluorouracil, capecitibine, gemcitabine, fludarabine, cytarabine, mercaptopurine, thioguanine, pentostatin, and hydroxyurea.

22. The method of any of claims 1 to 9, wherein the chemotherapeutic agent is a cell replication disrupting agent.

23. The method of claim 22, wherein the cell replication disrupting agent is selected from a group comprising paclitaxel, docetaxel, vincristine, vinblastin, thalidomide, lenalidomide, CC-5013, CC-4047, protein tyrosine kinase inhibitors, proteasome inhibitors, NF-κB inhibitors, and related analogs thereof.

24. The method of claim 23, wherein the protein tyrosine kinase inhibitor is selected from a group comprising imatinib mesylate and gefitinib.

25. The method of claim 23, wherein the proteasome inhibitors is bortezomib.

26. The method of claim 23, wherein the NF-κB inhibitor is an inhibitor of IκB kinase.

27. The method of claim 22, wherein the cell replication disrupting agent is an antibody.

28. The method of any one of claims 1 to 27, wherein the uterine cancer is endometrial cancer.

29. The method of any one of claims 1 to 28, further comprising administering to the patient an effective amount of at least one additional anti-cancer agent.

30. The method of claim 29, wherein the at least one additional anti-cancer agent is selected from the group consisting of platinum-based chemotherapy drugs, taxanes, tyrosine kinase inhibitors, anti-EGFR antibodies, anti-ErbB2 antibodies, and combinations thereof.

31. The method of claim 29, wherein the at least one additional anti-cancer agent comprises an EGFR inhibitor.

32. The method of claim 31, wherein the EGFR inhibitor comprises an anti-EGFR antibody.

33. The method of claim 32, wherein the anti-EGFR antibody comprises cetuximab.

34. The method of claim 32 wherein the anti-EGFR antibody is selected from the group consisting of matuzumab, panitumumab, and nimotuzumab.

35. The method of claim 31, wherein the EGFR inhibitor is a small molecule inhibitor of EGFR signaling.

36. The method of claim 35, wherein the small molecule inhibitor of EGFR signaling is selected from the group consisting of gefitinib, lapatinib, canertinib, pelitinib, erlotinib HCL, PKI-166, PD158780, and AG 1478.

37. The method of any of claims 1 to 36 wherein the anti-EMP2 antibody is conjugated with an effector moiety.

38. The method of claim 37, wherein the effector moiety is a toxic agent.

39. The method of claim 38, wherein the toxic agent is such as ricin.

40. The method of any one of claims 1 to 39, wherein the anti-EMP2 and the anti-angiogenic agent are administered simultaneously.

41. The method of any one of claims 1 to 39, wherein the anti-EMP2 and the anti-angiogenic agent are administered sequentially.

42. The method of any one of claims 1 to 39, wherein the anti-EMP2 and the chemotherapeutic agent are administered simultaneously.

43. The method of any one of claims 1 to 39, wherein the anti-EMP2 and the chemotherapeutic agent are administered sequentially.

44. The method of any of claims 1 to 43, wherein the anti-EMP2 antibodies are used in vaccine therapies for the cancer.

45. The method of any of claims 1 to 44, wherein the patient is a human or a mammal.

46. The method of any one of claims 1 to 45, further comprising a companion diagnostic.

47. The method of claim 46 wherein the companion diagnostic comprises an anti-EMP2 antibody.

48. The method of any one of claims 1 to 47, wherein the anti-EMP2 antibody is conjugated to a diagnostic moiety.

49. A method of treating a non-neoplastic condition, comprising administering an effective amount of an EMP2 inhibitor to a subject in need thereof.

50. The method of claim 49, wherein the anti-EMP2 antibody specifically binds to an epitope in the second extracellular loop of EMP2.

51. The method of claim 50, wherein the epitope in the second extracellular loop of EMP2 comprises the amino acid sequence DIHDKNAKFYPVTREGSYG.

52. The method of any of claims 49 to 51, wherein the anti-EMP2 antibody is a humanized or fully human antibody.

53. The method of any of claims 49 to 52, wherein the anti-EMP2 antibody is a diabody.

54. The method of any of claims 49 to 52, wherein the anti-EMP2 antibody is a triabody.

55. The method of any of claims 49 to 52, wherein the anti-EMP2 antibody is a minibody.

56. The method of any of claims 49 to 52, wherein the anti-EMP2 antibody is a single chain antibody.

57. The method of any of claims 49 to 56, wherein the effective amount of an anti-EMP2 antibody further comprises a physiological acceptable carrier or a pharmaceutically acceptable carrier.

58. The method of any of claims 49 to 57, wherein the anti-EMP2 antibody competes with an antibody comprising the heavy and light chain variable regions of a KS49, a KS41, a KS83, or a KS89 diabody.

59. The method of any of claims 49 to 57, wherein the antibody shares 90% amino acid identity with heavy and light chain variable regions of a KS49, a KS41, a KS83, or a KS89 diabody.

60. The method of any of claim 49 or 59, wherein the EMP2 inhibitor is a shRNA, a ribozyme, or an anti-EMP2 antibody.

61. The method of any of claims 49 to 60, wherein the EMP2 inhibitor is administered before, after or concomitantly with an anti-angiogenic agent.

62. The method of claim 61, wherein the anti-angiogenic agent is a VEGF inhibitor.

63. The method of claim 62, wherein the VEGF inhibitor comprises an anti-VEGF antibody.

64. The method of claim 63, wherein the anti-VEGF antibody is bevacizumab.

65. The method of claim 63, wherein the anti-VEGF antibody is selected from a group comprising pazopanib, sorafenib, sunitinib, vandeteanib, cabozantinib, ponatinib, axitinib, and aflibercept.

66. The method of any of claims 49 to 60, wherein the EMP2 inhibitor is administered before, after or concomitantly with a chemotherapeutic agent.

67. The method of claim 66, wherein the chemotherapeutic agent is a DNA damaging chemotherapeutic agent or a replication disrupting agent.

68. The method of any one of claims 49 to 67, further comprising administering to the patient an effective amount of at least one additional anti-cancer agent.

69. The method of claim 68, wherein the at least one additional anti-cancer agent is selected from the group consisting of platinum-based chemotherapy drugs, taxanes, tyrosine kinase inhibitors, anti-EGFR antibodies, anti-ErbB2 antibodies, and combinations thereof.

70. The method of claim 68, wherein the at least one additional anti-cancer agent comprises an EGFR inhibitor.

71. The method of claim 70, wherein the EGFR inhibitor comprises an anti-EGFR antibody.

72. The method of claim 71, wherein the anti-EGFR antibody comprises cetuximab.

73. The method of claim 71 wherein the anti-EGFR antibody is selected from the group consisting of matuzumab, panitumumab, and nimotuzumab.

74. The method of claim 70, wherein the EGFR inhibitor is a small molecule inhibitor of EGFR signaling.

75. The method of claim 74, wherein the small molecule inhibitor of EGFR signaling is selected from the group consisting of gefitinib, lapatinib, canertinib, pelitinib, erlotinib HCL, PKI-166, PD158780, and AG 1478.

76. The method of any of claims 49 to 75 wherein the anti-EMP2 antibody is conjugated with an effector moiety.

77. The method of claim 76, wherein the effector moiety is a toxic agent.

78. The method of claim 77, wherein the toxic agent is such as ricin.

79. The method of any of claims 49 to 78, wherein the anti-EMP2 antibodies are used in vaccine therapies for the cancer.

80. The method of any of claims 49 to 79, wherein the patient is a human or a mammal.

81. The method of any one of claims 49 to 80, further comprising a companion diagnostic.

82. The method of claim 81 wherein the companion diagnostic comprises an anti-EMP2 antibody.

83. The method of any one of claims 49 to 82, wherein the anti-EMP2 antibody is conjugated to a diagnostic moiety.

84. A method of any of claims 49 to 83, wherein the non-neoplastic condition comprises rheumatoid arthritis, psoriasis, atherosclerosis, diabetic retinopathy, retrolentral fibroplasia, thyroid hyperplasia, chronic inflammation, lung inflammation, nephrotic syndrome, preclampsia, ascites, pericardial effusion, or pleural effusion.


SEQUENCE LISTING (41 sequences)

SEQ ID NO: 1 - protein, 167 aa, Homo sapiens
MLVLLAFIIAFHITSAALLFIATVDNAWWVGDEFFADVWRICTNNTNCTVINDSFQEYSTLQAVQATMILSTILCCIAFFIFVLQLFRLKQGERFVLTSIIQLMSCLCVMIAASIYTDRREDIHDKNAKFYPVTREGSYGYSYILAWVAFACTFISGMMYLILRKRK

SEQ ID NO: 2 - protein, 20 aa, Artificial Sequence
  Description of Artificial Sequence synthetic second extracellular loop of human EMP2
EDIHDKNAKFYPVTREGSYG

SEQ ID NO: 3 - DNA, 1484 bp, Homo sapiens
gaggggccccgccgcctagagggtggagggagggcgcgcagtcccagcccagagcttcaaaacagcccggcggcctcgcctcgcacccccagccagtccgtcgatccagctgccagcgcagccgccagcgccggcacatcccgctctgggctttaaacgtgacccctcgcctcgactcgccctgccctgtgaaaatgttggtgcttcttgctttcatcatcgccttccacatcacctctgcagccttgctgttcattgccaccgtcgacaatgcctggtgggtaggagatgagttttttgcagatgtctggagaatatgtaccaacaacacgaattgcacagtcatcaatgacagctttcaagagtactccacgctgcaggcggtccaggccaccatgatcctctccaccattctctgctgcatcgccttcttcatcttcgtgctccagctcttccgcctgaagcagggagagaggtttgtcctaacctccatcatccagctaatgtcatgtctgtgtgtcatgattgcggcctccatttatacagacaggcgtgaagacattcacgacaaaaacgcgaaattctatcccgtgaccagagaaggcagctacggctactcctacatcctggcgtgggtggccttcgcctgcaccttcatcagcggcatgatgtacctgatactgaggaagcgcaaatagagttccggagctgggttgcttctgctgcagtacagaatccacattcagataaccattttgtatataatcattattttttgaggtttttctagcaaacgtattgtttcctttaaaagccaaaaaaaaaaaaaaaaaaaaaaaaaaaaaaaaaaaaaaaaaaaaaatccaaaagagagaagagtttttgcattcttgagatcagagaatagactatgaaggctggtattcagaactgctgcccactcaaaagtctcaacaagacacaagcaaaaatccagcaatgctcaaatccaaaagcactcggcaggacatttcttaaccatggggctgtgatgggaggagaggagaggctgggaaagccgggtctctggggacgtgcttcctatgggtttcagctggcccaagcccctcccgaatctctctgctagtggtgggtggaagagggtgaggtggggtataggagaagaatgacagcttcctgagaggtttcacccaagttccaagtgagaagcaggtgtagtccctggcattctgtctgtatccaaaccagagcccagccatccctccggtattggggtgggtcagaaaaagtctcacctcaatttgccgacagtgtcacctgcttgccttaggaatggtcatccttaacctgcgtgccagatttagactcgtctttaggcaaaacctacagcgcccccccctcaccccagacctacagaatcagagtcttcaagggatggggccagggaatctgcatttctaatgcgctccctgggcaacgcttca

SEQ ID NO: 4 - protein, 18 aa, Artificial Sequence
  Description of Artificial Sequencesynthetic linker between VL and VH of chimeric antibody
GSTSGGGSGGGSGGGGSS

SEQ ID NO: 5 - protein, 167 aa, Artificial Sequence
  Description of Artificial Sequencesynthetic EMP2 sequence
MLVILAFIIVFHIVSTAFISTIDNAWIVGDSADLRVCTNSTLCTEINELTGPEAFEGYSVWQAVQATMITILSSLCISFLIFLLQLFRLKQGERFVLTSIIQLMSCLCVMIGASIYTDRRQDLHQQNRKLYYLLQQGSYGYSFILAWVAFAFTFISGLMYMILRKRK

SEQ ID NO: 6 - protein, 123 aa, Artificial Sequence
  Description of Artificial Sequencesynthetic human anti-EMP2 antibody KS49 heavy chain variable region
MAQVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDRRGRKSAGIDYWGQGTLVTVSS

SEQ ID NO: 7 - protein, 124 aa, Artificial Sequence
  Description of Artificial Sequencesynthetic human anti-EMP2 antibody KS49 light chain variable region
DIQMTQSPSSLSASVGDRVTITCQASQDISNYLNWYQQKPGKAPKLLIYAASSLQSGVPSRFSGSGSGTDFTLTISSLQPEDFATYYCLQDYNGWTFGQGTKVDIKRAAAEQKLISEEDLNGAA

SEQ ID NO: 8 - protein, 122 aa, Artificial Sequence
  Description of Artificial Sequencesynthetic human anti-EMP2 antibody KS83 heavy chain variable region
MAQVQLVESGGGLVQPGGSLRLSCAASGFTFSSYAMHWVRQAPGKGLEWVAVISYDGSNKYYADSVKGRFTISRDNSKNTLYLQMNSLRAEDTAVYYCARTVGATGAFDIWGQGTMVTVSSS

SEQ ID NO: 9 - protein, 125 aa, Artificial Sequence
  Description of Artificial Sequence synthetic human anti-EMP2 antibody KS83 light chain variable region
DIVMTQSPSTVSASVGDRVIIPCRASQSIGKWLAWYQQKPGKAPKLLIYKASSLEGWVPSRFSGSGSGTEFSLTISSLQPDDSATYVCQQSHNFPPTFGGGTKLEIKRAAAEQKLISEEDLNGAA

SEQ ID NO: 10 - protein, 119 aa, Artificial Sequence
  Description of Artificial Sequence synthetic human anti-EMP2 antibody KS41 heavy chain variable region
MAQVQLVQSGGGLVQPGRSLRLSCAASGFSFSEYPMHWVRQAPGRGLESVAVISYDGEYQKYADSVKGRFTISRDDSKSTVYLQMNSLRPEDTAVYYCARTINNGMDVWGQGTTVTVSS

SEQ ID NO: 11 - protein, 124 aa, Artificial Sequence
  Description of Artificial Sequencesynthetic human anti-EMP2 antibody KS41 light chain variable region
DIVMTQSPSSLSASVGDRVTITCRASQGIRNDLGWYQQKPGKAPELLIYGASSLQSGVPSRFSGSGSGTDFTLTISSLQPEDSATYYCLQDYNGWTFGQGTKLEIKRAAAEQKLISEEDLNGAA

SEQ ID NO: 12 - protein, 119 aa, Artificial Sequence; identical to SEQ ID NO: 10
  Description of Artificial Sequence synthetic human anti-EMP2 antibody KS89 heavy chain variable region

SEQ ID NO: 13 - protein, 124 aa, Artificial Sequence; identical to SEQ ID NO: 11
  Description of Artificial Sequence synthetic human anti-EMP2 antibody KS89 light chain variable region

SEQ ID NO: 14 - protein, 5 aa, Artificial Sequence
  Description of Artificial Sequencesynthetic human anti-EMP2 antibody KS49 and KS83 heavy chain variable region CDR 1 Heavy (CDR-H1)
SYAMH

SEQ ID NO: 15 - protein, 5 aa, Artificial Sequence
  Description of Artificial Sequence synthetic human anti-EMP2 antibody KS41 and KS89 heavy chain variable region CDR 1 Heavy (CDR-H1)
EYPMH

SEQ ID NO: 16 - protein, 17 aa, Artificial Sequence
  Description of Artificial Sequence synthetic human anti-EMP2 antibody KS49 and KS83 heavy chain variable region CDR 2 Heavy (CDR-H2)
VISYDGSNKYYADSVKG

SEQ ID NO: 17 - protein, 17 aa, Artificial Sequence
  Description of Artificial Sequence synthetic human anti-EMP2 antibody KS41 and KS89 heavy chain variable region CDR 2 Heavy (CDR-H2)
VISYDGEYQKYADSVKG

SEQ ID NO: 18 - protein, 11 aa, Artificial Sequence
  Description of Artificial Sequence synthetic human anti-EMP2 antibody KS49 light chain variable region CDR 1 Light (CDR-L1)
QASQDISNYLN

SEQ ID NO: 19 - protein, 11 aa, Artificial Sequence
  Description of Artificial Sequence synthetic human anti-EMP2 antibody KS83 light chain variable region CDR 1 Light (CDR-L1)
RASQSIGKWLA

SEQ ID NO: 20 - protein, 11 aa, Artificial Sequence
  Description of Artificial Sequencesynthetic human anti-EMP2 antibody KS83 light chain variable region CDR 1 Light (CDR-L1)
RASQGIRNDLG

SEQ ID NO: 21 - protein, 7 aa, Artificial Sequence
  Description of Artificial Sequence synthetic human anti-EMP2 antibody KS49 light chain variable region CDR 2 Light (CDR-L2)
AASSLQS

SEQ ID NO: 22 - protein, 7 aa, Artificial Sequence
  Description of Artificial Sequences ynthetic human anti-EMP2 antibody KS83 light chain variable region CDR 2 Light (CDR-L2)
KASSLEG

SEQ ID NO: 23 - protein, 7 aa, Artificial Sequence
  Description of Artificial Sequence synthetic human anti-EMP2 antibody KS41 and KS89 light chain variable region CDR 2 Light (CDR-L2)
GASSLQS

SEQ ID NO: 24 - protein, 5 aa, Artificial Sequence
  Description of Artificial Sequencesynthetic short linker joining VH and VL domain
SGGGS

SEQ ID NO: 25 - DNA, 30 bp, Artificial Sequence
  Description of Artificial Sequence synthetic PCR forward primer for cloning EMP2
cgcggatcctctaccattgacaatgcctgg

SEQ ID NO: 26 - DNA, 33 bp, Artificial Sequence
  Description of Artificial Sequence synthetic PCR reverse primer for cloning EMP2
ccggaattcttacgcctgcatcacagaataacc

SEQ ID NO: 27 - protein, 4 aa, Artificial Sequence
  Description of Artificial Sequencesynthetic 4 amino acid long linker for biotinylation of scFv C- and N-termini
GSGS

SEQ ID NO: 28 - protein, 24 aa, Artificial Sequence
  Description of Artificial Sequence synthetic human EMP2 extracellular loop
DIHDKNAKFYPVTREGSTGGSGSK

SEQ ID NO: 29 - protein, 24 aa, Artificial Sequence
  Description of Artificial Sequence synthetic mouse EMP2 extracellular loop
DLHQQNRKLYYLLQEGSYGGSGSK

SEQ ID NO: 30 - protein, 6 aa, Artificial Sequence
  Description of Artificial Sequence synthetic 6 His scFv C-terminus tag
HHHHHH

SEQ ID NO: 31 - DNA, 48 bp, Artificial Sequence
  Description of Artificial Sequence synthetic 15 amino acid linker region to convert scFv fragments into diabody
agtggtggaggcggttcaggcggaggtggctctggcggtggcggatcg

SEQ ID NO: 32 - DNA, 15 bp, Artificial Sequence
  Description of Artificial Sequence synthetic shortened 5 amino acid linker region to convert scFv fragments into diabody
agtggtggaggatcg

SEQ ID NO: 33 - protein, 257 aa, Artificial Sequence
  Description of Artificial Sequence synthetic KS49 anti-EMP2 diabody
MAQVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDRRGRKSAGIDYWGQGTLVTVSSGGGSDIQMTQSPSSLSASVGDRVTITCQASQDISNYLNWYQQKPGKAPKLLIYAASSLQSGVPSRFSGSGSGTDFTLTISSLQPEDFATYYCLQDYNGWTFGQGTKVDIKRAAAEQKLISEEDLNGAAHHHHHH

SEQ ID NO: 34 - protein, 257 aa, Artificial Sequence
  Description of Artificial Sequence synthetic KS83 anti-EMP2 diabody
MAQVQLVESGGGLVQPGGSLRLSCAASGFTFSSYAMHWVRQAPGKGLEWVAVISYDGSNKYYADSVKGRFTISRDNSKNTLYLQMNSLRAEDTAVYYCARTVGATGAFDIWGQGTMVTVSSSGGGSDIVMTQSPSTVSASVGDRVIIPCRASQSIGKWLAWYQQKPGKAPKLLIYKASSLEGWVPSRFSGSGSGTEFSLTISSLQPDDSATYVCQQSHNFPPTFGGGTKLEIKRAAAEQKLISEEDLNGAAHHHHHH

SEQ ID NO: 35 - protein, 254 aa, Artificial Sequence
  Description of Artificial Sequence synthetic KS41 anti-EMP2 diabody
MAQVQLVQSGGGLVQPGRSLRLSCAASGFSFSEYPMHWVRQAPGRGLESVAVISYDGEYQKYADSVKGRFTISRDDSKSTVYLQMNSLRPEDTAVYYCARTINNGMDVWGQGTTVTVSSSGGGSDIVMTQSPSSLSASVGDRVTITCRASQGIRNDLGWYQQKPGKAPELLIYGASSLQSGVPSRFSGSGSGTDFTLTISSLQPEDSATYYCLQDYNGWTFGQGTKLEIKRAAAEQKLISEEDLNGAAHHHHHH

SEQ ID NO: 36 - protein, 257 aa, Artificial Sequence
  Description of Artificial Sequence synthetic KS89 anti-EMP2 diabody
MAQVQLVQSGGGLVQPGRSLRLSCAASGFSFSEYPMTHWVRQAPGRGLESVAVISYDGEYQKYADSVKGRFTISRDDSKSTVYLQMNSLRPEDTAVYYCARTINNGMDVWGQGTTVTVSSSGGGSDIVMETTQSPSSLSASVGDRVTITCRASQGIRNDLGWYQQKPGKAPELLIYGASSLQSGVPSRFSGSGSGTDFTLTISSLQPEDSATYYCLQDYNGWTFGQGTKLEIKRAAAEQKLISEEDLNGAAHHHHHH

SEQ ID NO: 37 - protein, 10 aa, Artificial Sequence
  Description of Aritifical Sequence synthetic human anti-EMP2 antibody KS83 heavy chain variable region CDR 3 Heavy (CDR-H3)
TVGATGAFDI

SEQ ID NO: 38 - protein, 9 aa, Artificial Sequence
  Description of Artificial Sequence synthetic human anti-EMP2 antibody KS83 light chain variable region CDR 3 Light (CDR-L3)
QQSHNFPPT

SEQ ID NO: 39 - protein, 12 aa, Artificial Sequence
  Description of Artificial Sequence synthetic human anti-EMP2 antibody KS49 heavy chain variable region CDR 3 Heavy (CDR-H3)
DRRGRKSAGIDY

SEQ ID NO: 40 - protein, 8 aa, Artificial Sequence
  Description of Artificial Sequence synthetic human anti-EMP2 antibody KS41, KS49, and KS89 light chain variable region CDR 3 Light (CDR-L3)
LQDYNGWT

SEQ ID NO: 41 - protein, 8 aa, Artificial Sequence
  Description of Artifical Sequence synthetic human anti-EMP2 antibody KS41 and KS89 heavy chain variable region CDR 3 Heavy (CDR-H3)
TINNGMDV